(19) United States
(12) Patent Application Publication
(10) Pub. No.: US 2013/0184636 A1
(43) Pub. Date: Jul. 18, 2013

(54) System and Method for Controlling Neural and Muscular Function

(71) Applicant: Graham Creasey, Palo Alto, CA (US)

(72) Inventor: Graham Creasey, Palo Alto, CA (US)

(21) Appl. No.: 13/740,198

(22) Filed: Jan. 12, 2013

Related U.S. Application Data
(60) Provisional application No. 61/586,478, filed on Jan. 13, 2012.

(51) Int. Cl. A61N 5/06

(52) U.S. Cl. CPC A61N 5/062
USPC 604/20

(57) ABSTRACT

A system and method for controlling neural and muscular function is disclosed in which opsins are introduced into a neural circuit such that the control of optical signals transmitted to the opsins results in the control of neural and muscular functions. Specifically disclosed is the control of the bladder, bowel, and sexual functions of a human.

### CROSS REFERENCE TO RELATED APPLICATIONS

This application claims the benefit of U.S. Provisional Patent Application No. 61/586,478 filed on Jan. 13, 2012 entitled “System and Method for Controlling Neural and Muscular Function,” the disclosure of which is hereby incorporated by reference.

This application is related to U.S. patent application Ser. No. 12/579,581 filed on Oct. 15, 2009, titled “Systems and Methods for Selectively Stimulating Components In, On, or Near the Pudendal Nerve or Its Branches to Achieve Selective Physiologic Responses,” the disclosure of which is hereby incorporated by reference.

This application is related to U.S. Pat. No. 6,907,293 filed on Mar. 29, 2002, titled “Systems and Methods for Selectively Stimulating Components in, on, or near the Pudendal Nerve or its Branches to Achieve Selective Physiologic Responses,” the disclosure of which is hereby incorporated by reference.

This application is related to U.S. Pat. No. 5,199,430 filed on Mar. 11, 1991, titled “Micturitional Assist Device,” the disclosure of which is hereby incorporated by reference.

### BACKGROUND OF THE INVENTION

This invention relates in general to the control of neural and muscular function, such as in animals. Embodiments of this invention relate to the restoration of neural and muscular functions in humans.

Injury or disease of nerves impairs the functions that they control, specifically contraction of muscles, sensation and secretion. In the case of nerves in the central nervous system (brain and spinal cord) of a living human, this damage and loss of function is usually permanent.

Function can sometimes be restored by interacting with remaining nerves chemically or electrically. Pharmaceuticals or electrical devices similar to pacemakers can modify existing function, a process sometimes known as neuromodulation. Lost function can sometimes be restored using devices known as neural prostheses, a process known as functional electrical stimulation.

Specifically, paralyzed muscles can be made to contract by stimulating their motor nerves directly or by stimulating sensory nerves to produce reflex contraction.

Spastic muscles can sometimes have their contraction reduced by stimulating sensory nerves to inhibit contraction, or by blocking motor or sensory nerves using specific forms of chemical or electrical intervention.

Sensation, including pain, can be reduced by chemical or electrical interventions to reduce conduction in sensory nerves or in nerve cells, tracts, and circuits in the central nervous system.

Secretion can be increased or decreased by chemical or electrical interaction with nerves controlling cells which release secretions through the skin (e.g. sweat), into body cavities (e.g. stomach acid), the bloodstream (e.g. hormones), or the tissues (e.g. neurotransmitters, cytokines and other molecules).

Current art teaches using these methods of chemical or electrical interaction to provide some level of control to neural and muscle function. However, electrical intervention usually has limited specificity. Many attempts are made to improve specificity by local application of stimuli or by design of electrodes and stimulus parameters, but there remain fundamental limitations due to the anatomy and electrophysiology of nerves. As well, electrical interaction with nerves is primarily stimulatory, although some electrical techniques can inhibit the generation or conduction of electrical activity in nerves.

Additionally, chemical intervention usually has limited specificity, particularly when pharmaceuticals are given systemically. Many attempts are made to improve specificity by local application or release of pharmacologically active substances or by developing more specific medications, but there remain fundamental limitations due to factors such as similarity between physiological receptors in different tissues. While chemical interaction with nerves can be stimulatory or inhibitory, the difficulties presented by chemical interaction's limited specificity makes it an ineffective tool in many desired applications.

### SUMMARY OF THE INVENTION

This invention relates to the control of neural and muscular function in a living body. This invention also relates to the restoration of neural and muscular functions in a human.

Various advantages of this invention will become apparent to those skilled in the art from the following detailed description of the preferred embodiments, when read in light of the accompanying drawings.

### DESCRIPTION OF THE DRAWINGS

The drawings described herein are for illustration purposes only and are not intended to limit the scope of the present disclosure in any way. None of the representations or diagrams included with this disclosure is intended to represent actual scale or relative scale of the objects they are representing.

FIG. 1 is a Venn diagram representation of a method of achieving selectivity in optogenetic intervention.

FIG. 2a is a diagrammatic representation of a method of achieving selective activation of a first set of nerves.

FIG. 2b is a diagrammatic representation of a method of achieving selective activation of a second set of nerves.

FIG. 2c is a diagrammatic representation of a method of achieving selective activation of a first set of nerves and inactivation of a second set of nerves.

FIG. 2d is a diagrammatic representation of a method of achieving selective inactivation of the first set of nerves and activation of the second set of nerves.

FIG. 3 is a diagrammatic representation of an opsin site showing a cellular membrane containing opsins.

FIG. 4 is a diagrammatic representation of a portion of a nervous system treated by opsins.

FIG. 5 is a schematic flow diagram of an embodiment of a method of introducing opsins.

FIG. 6 is a diagrammatic representation of an opsin-affected neural circuit.

FIG. 7a is a diagrammatic representation of a light source applying an optical signal directly to an opsin site.

FIG. 7b is a diagrammatic representation of an optical signal being conducted to an opsin site.

FIG. 7c is a diagrammatic representation of a light modifying device creating an optical signal from pre-optical-signal light, the optical signal being then conducted to an opsin site.

FIG. 7d is a diagrammatic representation of a light modifying device creating an optical signal from pre-optical-signal light, the optical signal being then conducted to an opsin site.

FIG. 8 is a schematic flow diagram of optogenetic intervention with sensory feedback.

FIG. 9a is a diagrammatic representation of an embodiment of an optogenetic intervention system installed on a nerve bundle.

FIG. 9b is a diagrammatic representation of an embodiment of an optogenetic intervention system installed on a nerve bundle.

FIG. 10 is a diagrammatic representation of an embodiment of an optogenetic intervention system as used to control bladder contraction.

FIG. 11 is a diagrammatic representation of embodiments of optogenetic intervention systems installed on nerve bundles affecting bladder, bowel and sexual function.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Molecules have been discovered or developed which are capable of changing form and/or performing certain actions based on the application of light. These light-sensitive molecules, known as opsins, are capable of transporting ions across a cellular membrane. Opsins are capable of influencing the electrical activity of nerves. Opsins may affect a nerve in various ways, such as by increasing or decreasing the electrical activity of that nerve. Precise control over the electrical activity of a nerve can be precisely controlled by altering the intensity, timing, wavelength, and duration of light applied to the opsins. While certain opsins are known at this point, the disclosed method and system can be used with heretofore undiscovered or undeveloped opsins. The techniques of using opsins and applied light to control the electrical activity of a nerve may be described as optogenetic intervention. The use of optogenetic intervention allows for greater specificity in the control of nerves than is currently available with other known technologies.

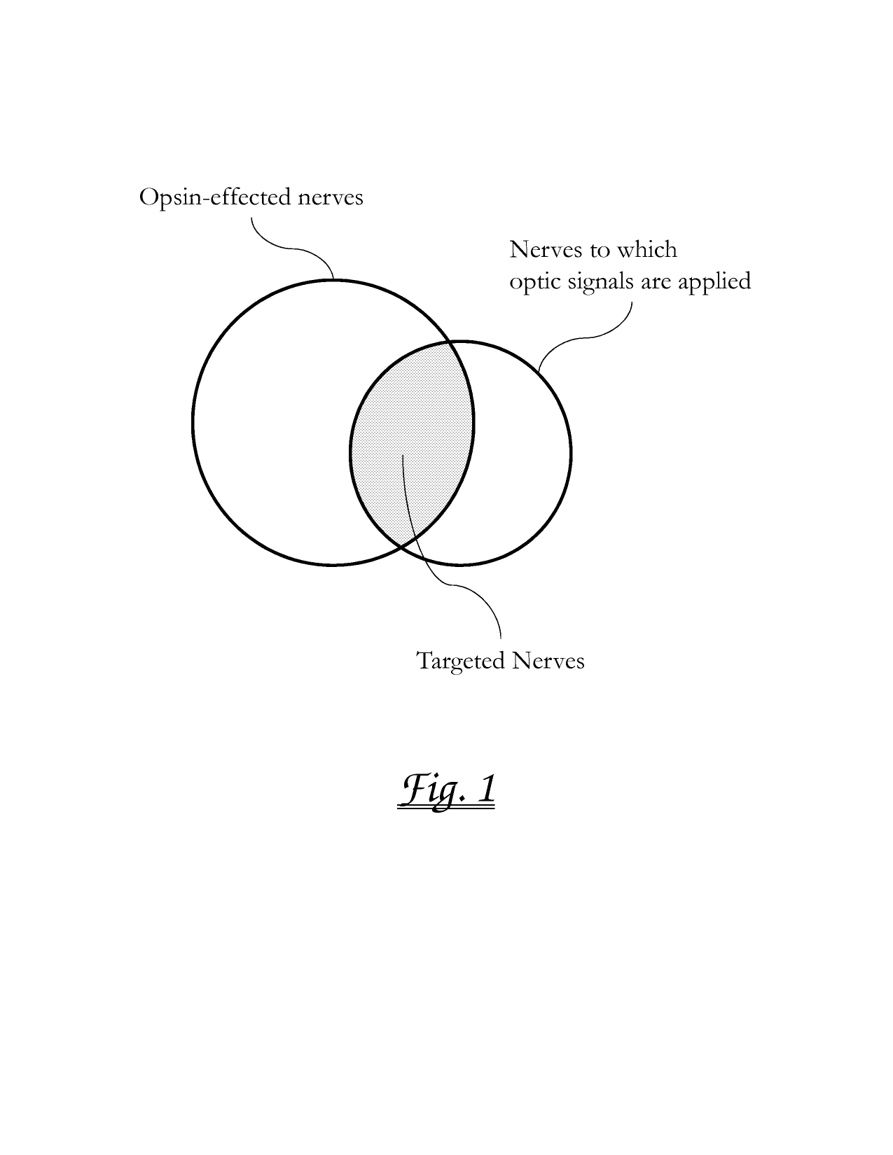

Opsins are themselves controlled by controlling the amount of light applied to the opsins. Light can be introduced into specific peripheral nerves or parts of nerves, or specific parts of the central nervous system, according to the method of introduction. Thus, the intersection of selectively introduced opsins with selectively applied light enables highly selective activation or inactivation of nerves. As shown in FIG. 1, high selectivity can be achieved by applying light to only a portion of the nervous system which has been affected by opsins, thereby only activating the desired, targeted nerves.

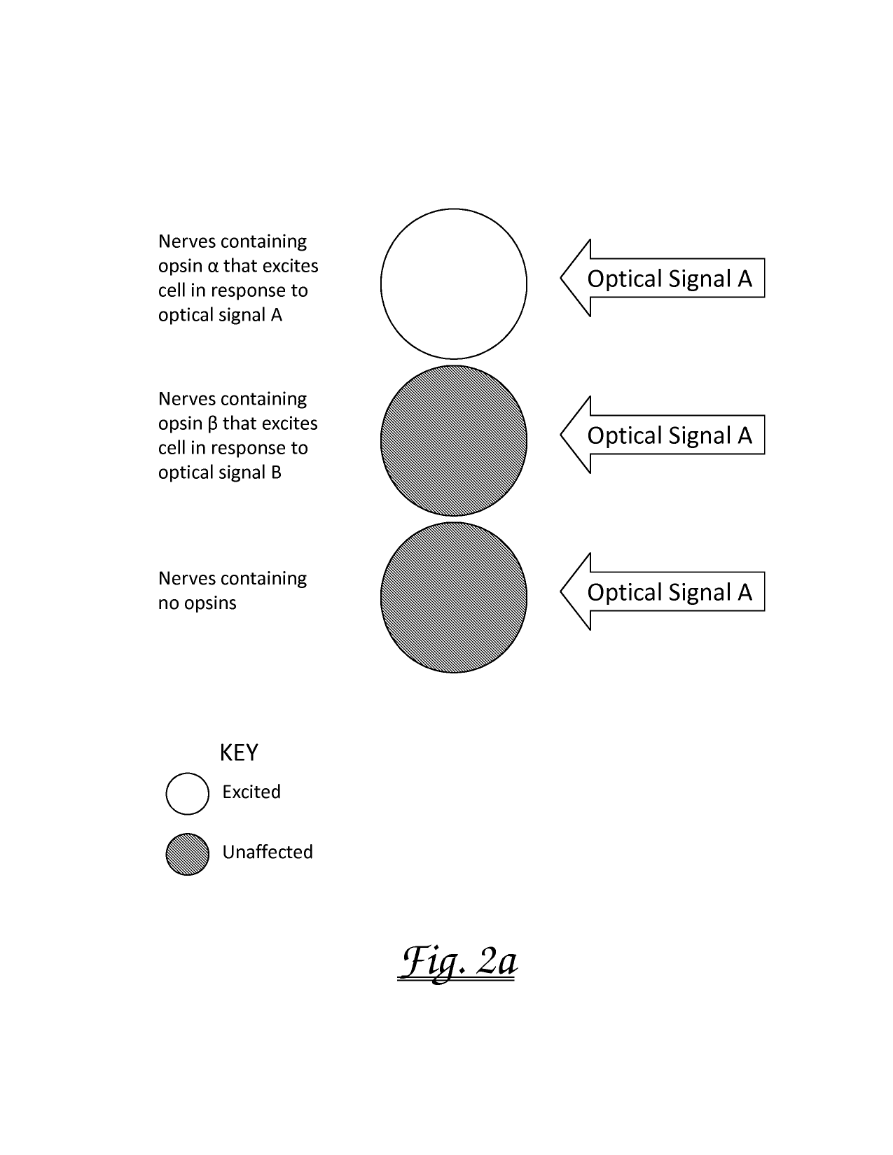

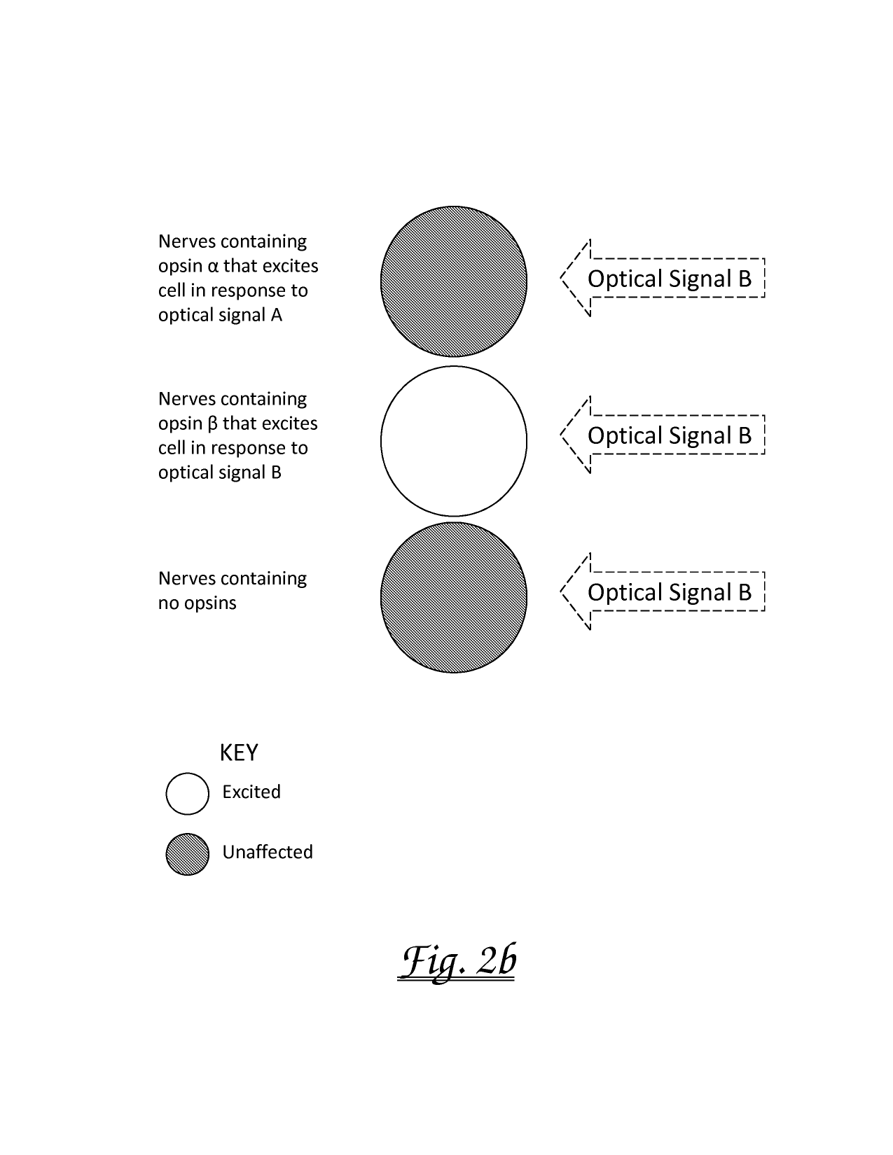

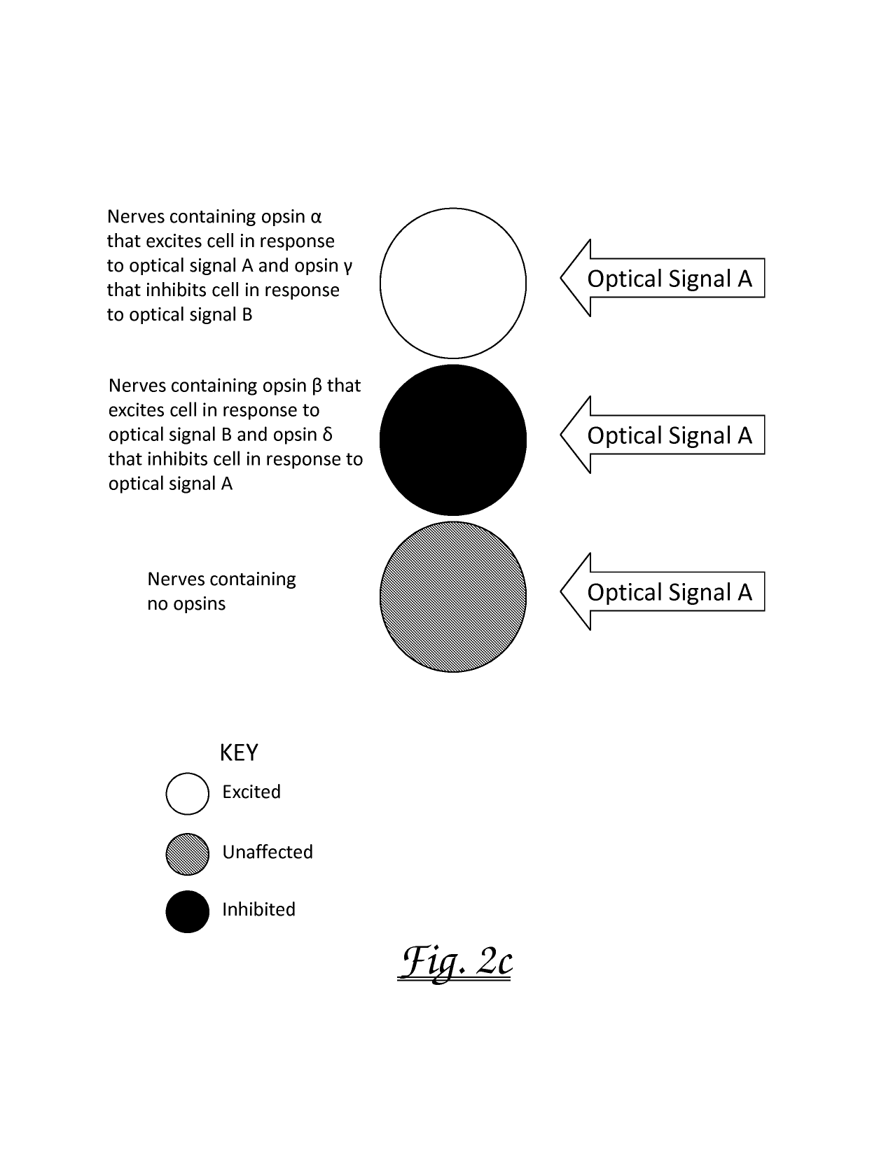

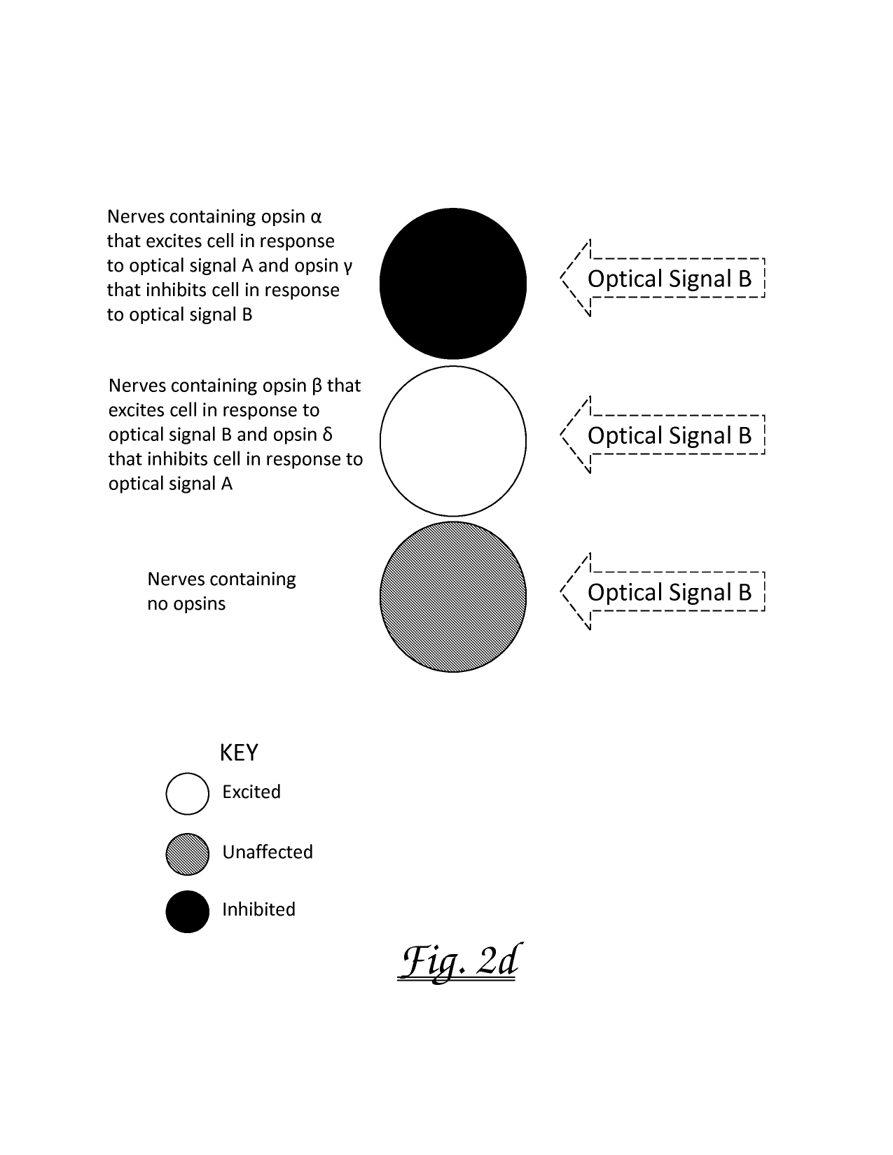

The use of optogenetic intervention further allows for great control in the inhibition of electrical activity of a nerve. Because different opsins can cause different ions to build up on different sides of a membrane, opsins that reduce electrical activity of a nerve can be selected for use in certain occasions. Therefore, optogenetic interaction can inhibit generation or conduction of electrical activity in specific nerves or circuits for various durations in response to application of light. In fact, opsins that reduce electrical activity and opsins that increase electrical activity can be co-located near one another and even within the same membrane, but may be triggered by different optical signals, thereby allowing for further selectivity. Referring to FIG. 2a, three subsets of nerves are shown from top to bottom, one affected with opsin α, one affected with opsin β, and one not affected with opsins. Opsin α excites its cell in response to optical signal A and opsin β excites its cell in response to optical signal B. The application of optical signal A to each of the subsets of nerves only triggers the top subset of nerves, those containing opsin α, causing an excitation of those nerves. Since opsin β is not responsive to optical signal A, it is unaffected, similarly to the subset of nerves containing no opsins. Referring to FIG. 2b, three subsets of nerves are shown similar to those of FIG. 2a, with subsets of nerves affected by, from top to bottom, opsin α, opsin β, and no opsins. Opsins α and β behave as in FIG. 2a. Since opsin α is not responsive to optical signal B, the application of optical signal B to each subset of nerves will only trigger the middle subset of opsin-affected nerves, those containing opsin δ, causing an excitation of those nerves. The subset of nerves containing no opsins remains unaffected by any optical signal. As used above with reference to FIGS. 2a-2b, opsins may be selected such that the application of an optical signal causes inhibition rather than excitation.

In an embodiment, excitation opsins and inhibition opsins may be introduced into the same cell, but responsive to different optical signals. In such embodiments, when a first optical signal is transmitted to the opsin site, the result may be an excitation, but when a second optical signal is transmitted to the opsin site, the result may be an inhibition. Referring to FIG. 2c, three subsets of nerves are shown, from top to bottom, one containing both opsin α and opsin γ, one containing both opsin β and opsin δ, and one containing no opsins. Opsins α and β behave as described above with reference to FIGS. 2a and 2b. Opsin γ inhibits the cell in response to optical signal B and opsin δ inhibits the cell in response to optical signal A. Application of optical signal A to each subset of nerves will cause the top subset to be excited due to the presence of opsin α, cause the middle subset to be inhibited due to the presence of opsin δ, and the bottom subset will remain unaffected. Referring to FIG. 2d, three subsets of nerves are shown, from top to bottom, one containing both opsin α and opsin γ, one containing both opsin β and opsin δ, and one containing no opsins. Opsins α, γ, β, and δ all behave as described above with reference to FIG. 2c. Application of optical signal B to each subset of nerves results in the top subset being inhibited due to the presence of opsin γ, the middle subset being excited due to the presence of opsin β, and the bottom subset remaining unaffected. Additionally, in an embodiment, specific opsins can be chosen such that activation of a first set of opsins may enable or disable the ability for a second set of opsins to be activated.

It will be understood that further combinations of specific opsins and specific optical signals can create further permutations and allow for increased selectivity.

In an embodiment similar to that shown in FIGS. 2c-d, optical signal A may be used to facilitate micturition and optical signal B may be used to facilitate continence. In such an embodiment, opsins may be selected such that the application of optical signal A would cause excitation of some nerves (e.g., nerves controlling bladder contractions) and simultaneously inhibition other (e.g., nerves controlling urethral sphincters) to produce voiding. Similarly, opsins may be selected such that the application of optical signal B would cause excitation of some nerves (e.g., nerves controlling urethral sphincters) and simultaneously inhibit others (e.g., nerves controlling bladder contractions).

Opsin-coding genes are specific genes which control the production of opsins. Opsin-coding genes can be introduced into cells, such as through a viral vector. Once introduced into a cell, the opsin-coding genes would cause the cell to begin to produce opsins. As used herein, the term opsin-coding genes refers to one or more genes which control the production of opsins.

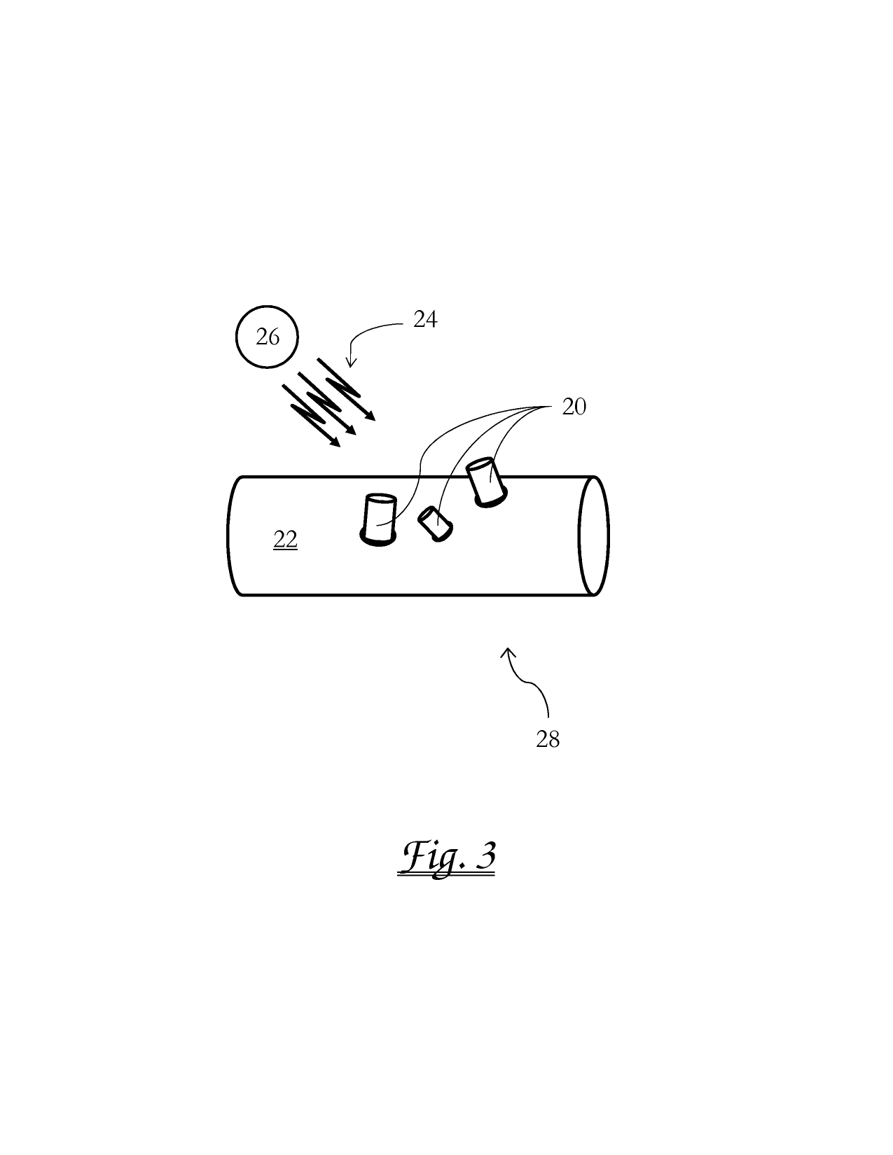

As shown in FIG. 3, once a cell begins to produce opsins 20, it may begin incorporating those opsins 20 into the cell's cellular membrane 22. Once a cell has incorporated opsins 20 into its cellular membrane 22, an optical signal 24 applied near the opsins 20 may increase or decrease the electrical activity of that cell. The optical signal 24 may be applied from a light source 26. In this fashion, an opsin-affected cell 28, such as a nerve cell, can be excited or inhibited on command through the judicious application of optical signals 24. In embodiments where the opsin-affected cell 28 is a nerve cell, control of an opsin-affected cell 28 further allows for control of the nerve circuit of which that opsin-affected cell 28 is part. Control of the opsin-affected cell 28 potentially allows for control of any directly or indirectly connected nerves or muscle cells.

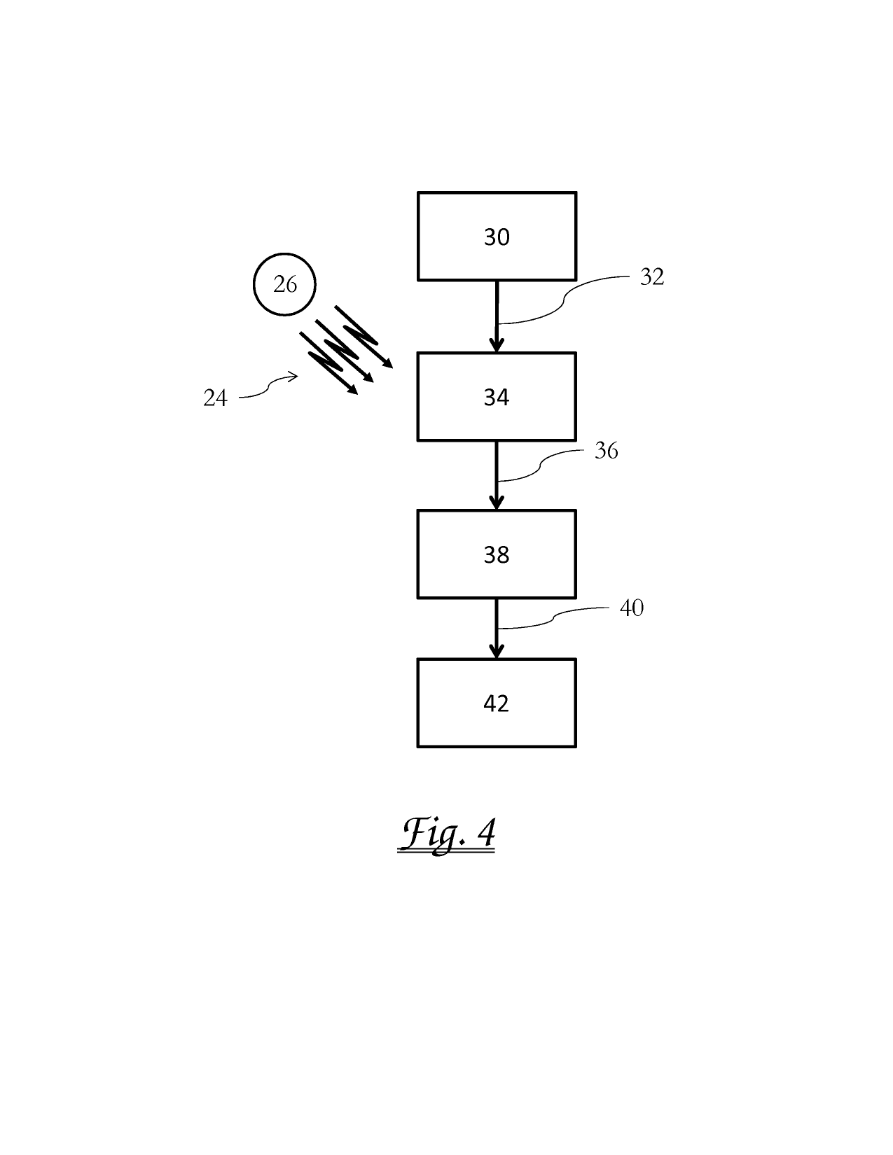

As shown in FIG. 4, an upstream nerve cell 30 sends an upstream activation signal 32 to an opsin-affected nerve cell 34. The opsin-affected nerve cell 34, upon receiving the upstream activation signal 32, will transmit a downstream activation signal 36 to a receiving nerve cell 38. Upon receiving the downstream activation signal 36, the receiving nerve cell 38 may transmit a muscle activation signal 40 to a muscle cell 42. This type of functionality regularly occurs between nerves and muscles which are not opsin-affected (i.e. the opsin-affected nerve cell 34 would instead be a regular nerve cell). In an embodiment containing an opsin-affected nerve cell 34, however, an optical signal 24 can be transmitted from a light source 26 to the opsin-affected nerve cell 34 to affect the opsin-affected nerve cell 34. In some embodiments, reception of an optical signal 24 may cause the opsin-affected nerve cell 34 to generate a downstream activation signal 36 which is then received by the receiving nerve cell 38. The receiving nerve cell 38 and any cells downstream of that cell may be agnostic as to whether the downstream activation signal 36 was a result of an optical signal 24 or an upstream activation signal 32. In some embodiments, transmission of an optical signal 24 to the opsin-affected nerve cell 34 may inhibit the opsin-affected nerve cell 34 from generating a downstream activation signal 36 in response to an upstream activation signal 32. It will be understood that the above example deals generally with nerves that activate downstream nerves in an excitatory sense, but that the principles of optogenetic intervention as disclosed herein may be applied equally to nerves which regularly inhibit downstream nerves or a combination thereof.

I. Introduction of Opsin-Coding Genes

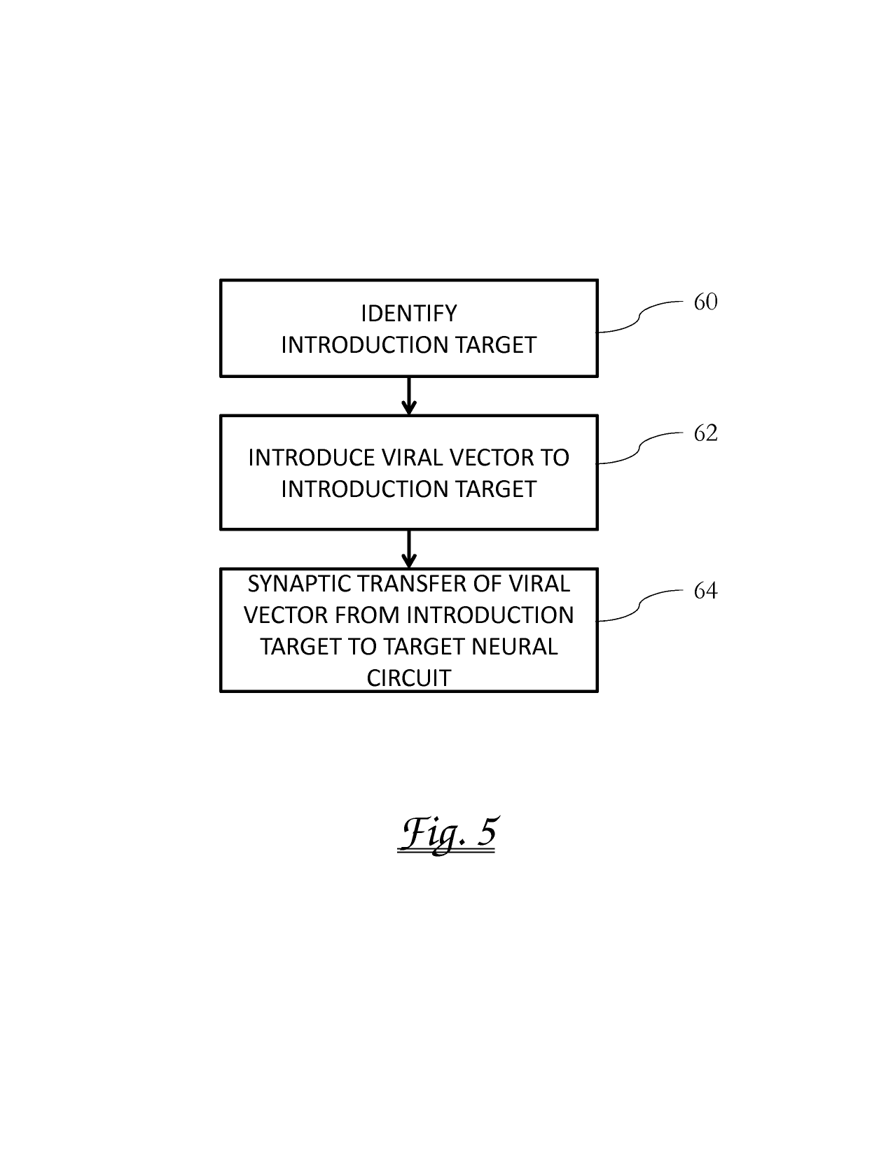

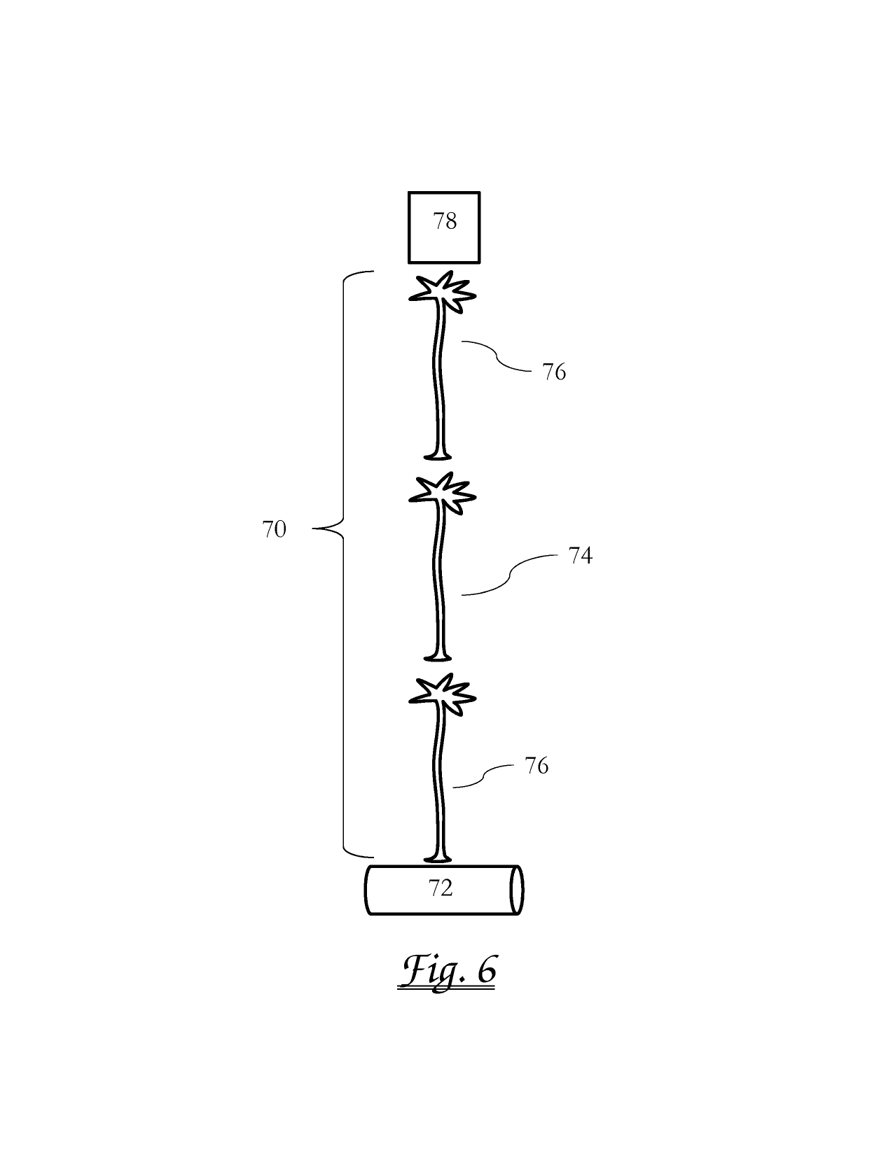

Introduction of opsin-coding genes can be accomplished through the introduction of viral vectors. As shown in FIGS. 5 and 6, a method of introducing opsin-coding genes to a target neural circuit 70 is shown. The target neural circuit 70 is a series or other collection of interconnected nerve cells which all control a desired function. An exemplary target neural circuit 70 may consist of all nerve cells in series with the motor neurons controlling the urethral sphincter muscles 72. As shown in FIG. 6, a target neural circuit 70 may consist of a plurality of nerve cells which are capable of propagating an activation signal from a source 78 to a receiver 72. In an exemplary embodiment, the source 78 may be the central nervous system and the receiver 72 may be a muscle. In other exemplary embodiments, the source 78 may be a sensory cell or sensory nerve within the body and the receiver 72 may be a nerve cell. Referring back to FIGS. 5 and 6, at step 60, the introduction target 74, is identified. The introduction target 74 may be one or more nerve cells that are a part of the target neural circuit 70. An exemplary introduction target 74 is a nerve cell that is in series with the motor neurons controlling the urethral sphincter muscles 72. At step 62, a viral vector 10 containing opsin-coding genes is introduced into the introduction target 74, such as via injection. At step 64, after the viral vector 10 is introduced into the introduction target 74, a certain period of time is left to elapse so that the viral vector 10 may be transferred to the other nerve cells 76 of the target neural circuit 70 by synaptic transfer, retrograde transport, and/or other mechanisms. In an exemplary embodiment, the introduction target 74 is a nerve cell which is located at a location remote from neurons and neural circuits for which optogenetic intervention is not desired. Through this introduction method, opsins 20 can be introduced to an entire neural circuit with a reduced possibility of “contaminating” neurons and neural circuits for which optogenetic intervention is not desired. Additionally, this introduction method may allow introduction of opsins into an entire neural circuit where a majority of the neural circuit is generally difficult-to-access, but where at least one neuron is reasonably accessible.

Using the aforementioned introduction method, genes can be introduced into specific peripheral nerves or parts of nerve, allowing for some viral vectors to be transferred between nerves, for example at a synapse, so that genes may be introduced into specific multicellular circuits in the peripheral or central nervous system.

II. Opsin Placement

Genes to control the production of opsins can be introduced with a viral vector by various means, such as the one outlined above. Alternatively, genes to control the production of opsins can be introduced in other ways. These genes may be injected into muscles whose control is desired in a fashion analogous to that described above. These genes may be distributed by retrograde transport into and through the nerves controlling those muscles. It will be understood that genes may be injected into nerves, ganglia, nerve trunks, nerve tracts or nerve centers whose control is desired.

III. Application of Light

Opsins operate by responding to light. Changes in the intensity, duration, timing, and frequency of any applied light, including changes in the presence and absence of light, may cause an opsin to affect the cell to which it is attached in different ways. As used herein, the term optical signal 24 may refer to any type of light signal, including but not limited to constantly applied light and pulsating light. As used herein, an optical signal 24 may refer to transmitted light having specific profiles for intensity, duration, timing, and wavelength.

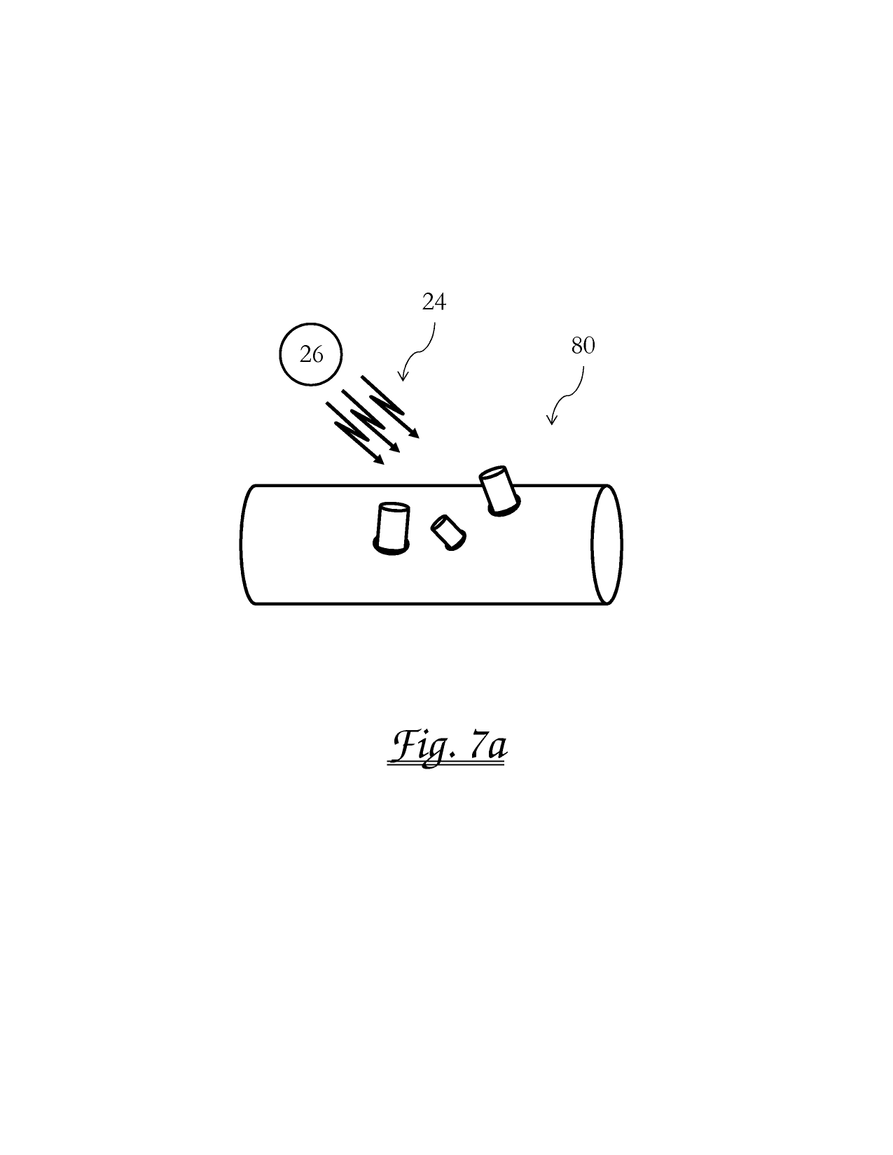

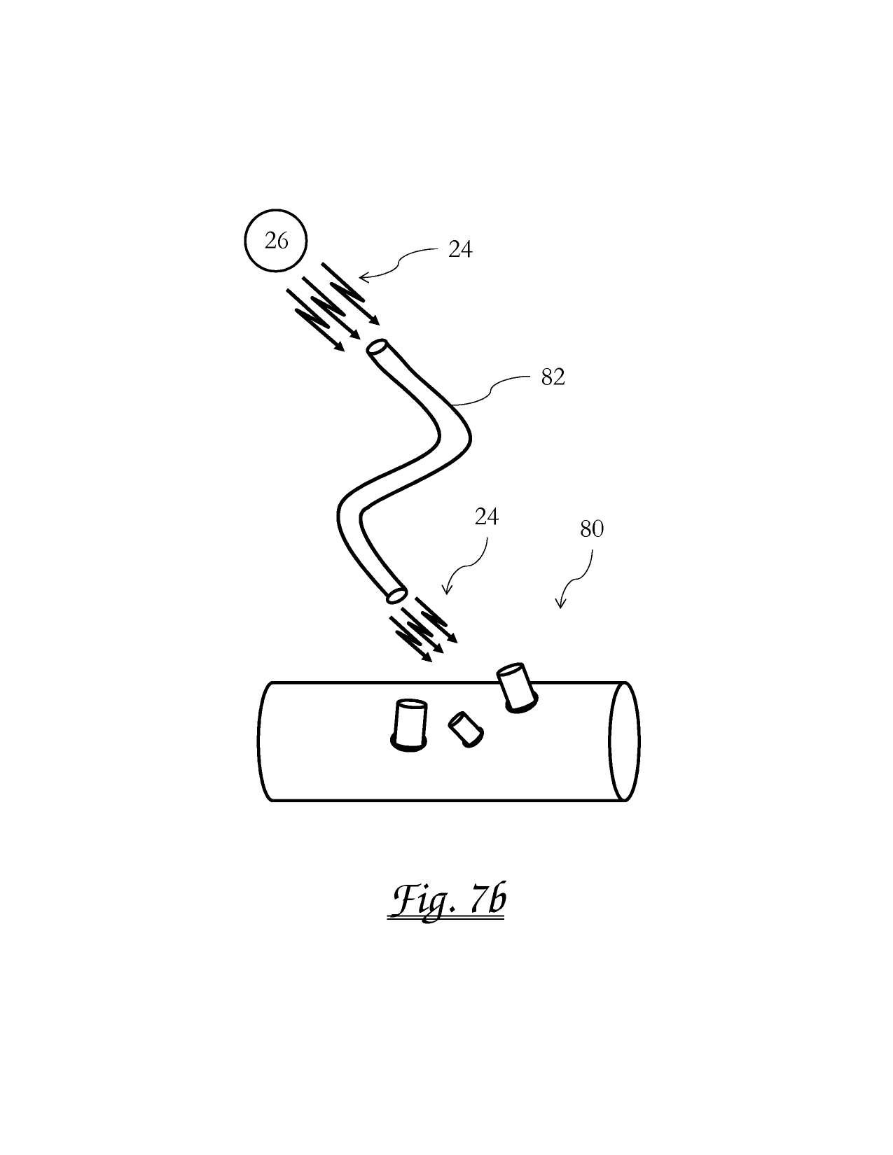

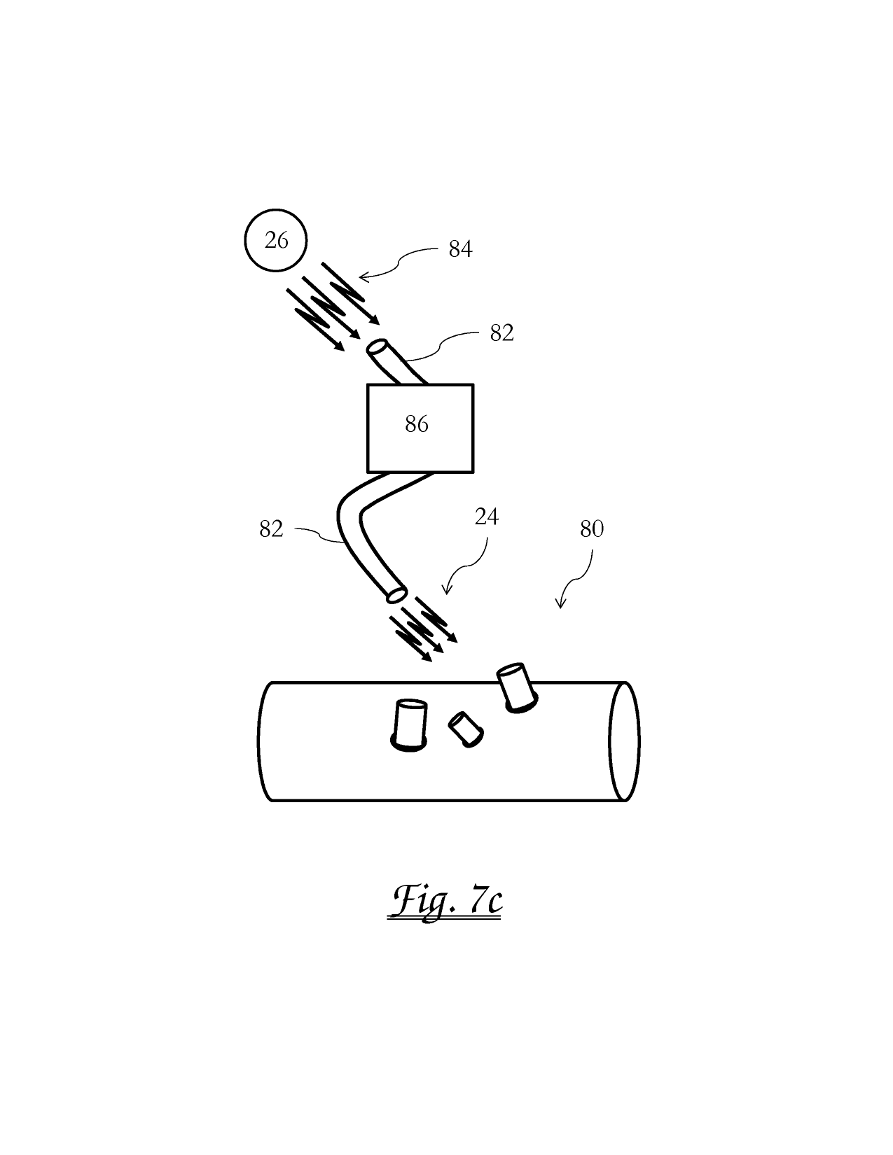

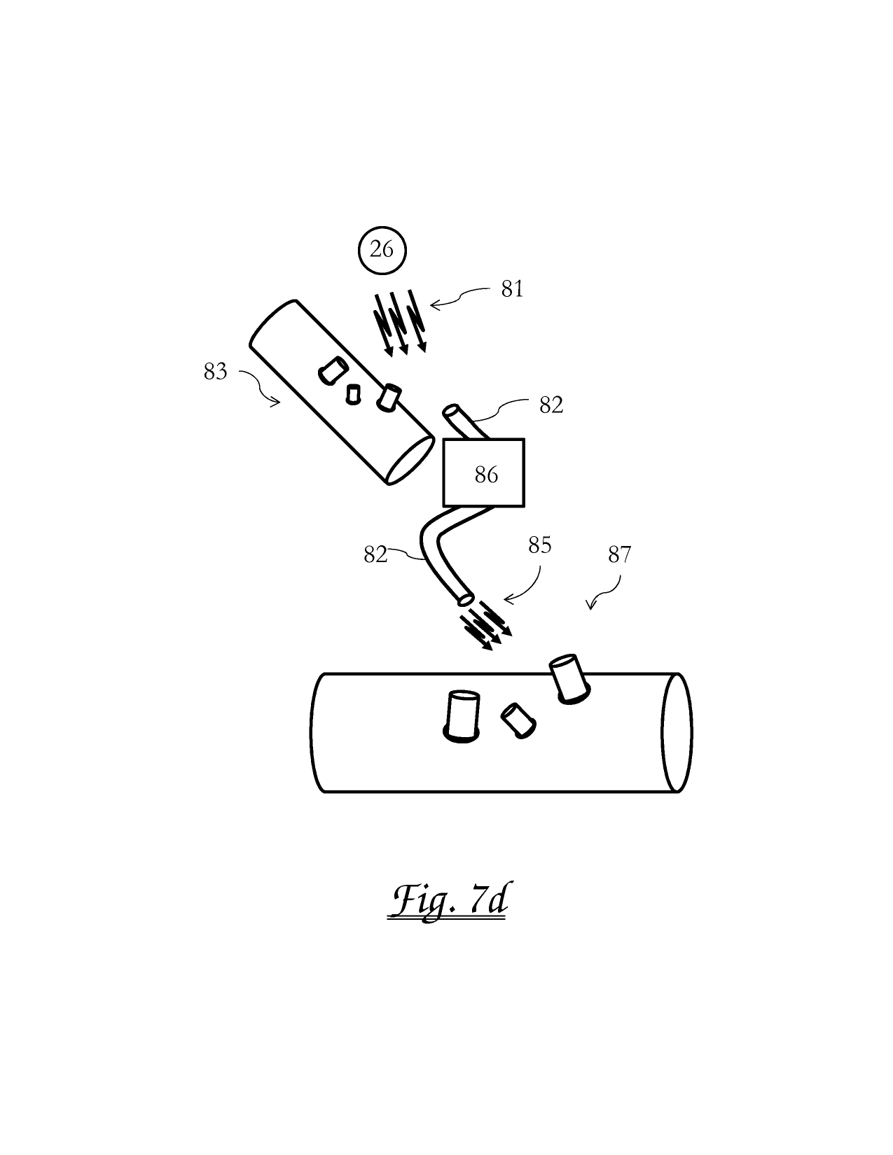

As shown in FIGS. 7a-7d, optical signals 24 can be introduced to an opsin site 80 by various methods. An opsin site 80 is a location containing opsins located in a cell membrane. In an embodiment shown in FIG. 7a, optical signals 24 can be introduced to the opsin site 80 directly by placing one or more light sources 26 on, in, or near the nerve tissue containing the desired opsin. Such light sources 26 are light emitting devices which may include electronic devices (e.g., light emitting diodes), phosphorescent or fluorescent materials (e.g., quantum dots and phosphors), or any other device or material that may be used to emit light.

In an embodiment shown in FIG. 7b, light may be applied to opsin sites by conducting optical signals 24 from one or more light sources 26 through the use of an optic conductor 82. In these embodiments, an optical signal 24 is created at a light source 26, such as a light emitting device described above, and that optical signal 24 is conducted through an optic conductor 82 to the opsin site 80. In one such embodiment, optical signals 24 are conducted to the opsin site 80 through optical fibers which may be placed on, in, or near the desired nerve tissue. In another such embodiment, optical signals 24 are conducted through translucent or transparent body tissues and fluids which are capable of conducting those optical signals 24 to the opsin sites 80 on the desired nerves. Such translucent or transparent body tissues and fluids may include the cerebrospinal fluid surrounding the brain, spinal cord, and nerve roots. Optical signals can be passed to many nerves (e.g., sacral nerves and nerve roots in cauda equina) in this fashion. Therefore, in some embodiments, the optical conductor 82 is comprised of one or more body tissues or fluids. In some embodiments, the optical conductor 82 may be comprised of a material that is injected or implanted into the body on, in, or near an opsin site 80. In some embodiments, multiple optical conductors 82 may be used.

In certain embodiments, as shown in FIG. 7c, it is not necessary that the light source 26 produce the final optical signal that will be delivered to the opsin site, therefore allowing the use of more devices or materials as effective light sources 26. In such embodiments, the light source 26 may be a light emitting device as described above, or any other light source (e.g., sunlight, room light, or other external light). In such embodiments, the light source 26 may emit pre-optical-signal light 84 directly into a light modifying device 86, or the light source 26 may emit pre-optical-signal light 84 into an optical conductor 82 which is optically coupled to a light modifying device 86. In certain embodiments, the light modifying device 86 is capable of modifying the pre-optical-signal light 84 into the desired optical signal 24. In certain embodiments, the optical signal 24 would be conducted from the light modifying device 86 to the opsin site 80 through the optic conductor 82. In other embodiments not shown, the light modifying device 86 would be positioned near the opsin site 80 such that the optical signal 24 can be transmitted directly to the opsin site 80 from the light modifying device 86.

Optical signals 24 provided to an opsin site 80 can be controlled in various ways to ensure desired activation or inactivation of opsins 20. Optical signals 24 are signals which may include the application and/or withholding of light. The light provided in an optical signal 24 may vary in intensity, duration, timing and wavelength. As used in the claim, the qualities intensity, duration, timing, and wavelength can be of any value, including zero. Combinations of light application and withholding may make up a single optical signal 24. The application and/or withholding of light of a certain intensity, duration, timing and wavelength represents a single light profile. An optical signal 24 may be comprised of several simultaneous and/or sequential light profiles.

In certain embodiments, the light source 26 is designed to output an optical signal 24 which contains a specifically desired light profile designed to control certain opsins 20. The light source 26 may incorporate or be coupled to a microprocessor capable of controlling the light profile as emitted from the light source. As used herein, the term microprocessor may refer to any type of processor and regardless of size of the processor.

In certain embodiments, a light modifying device 86 may incorporate or be coupled to a microprocessor capable of controlling how the light modifying device 86 modifies the pre-optical-signal light 84. In these embodiments, the light modifying device 86 is capable of modifying the pre-optical-signal light 84 into an optical signal 24.

In some embodiments, the pre-optical-signal light 84 is an optical signal 24 originating from either another light modifying device 86 or a light source 26. The light modifying device 86 may be capable of altering at least one of the intensity, duration, timing and wavelength of the pre-optical-signal light 84 to create a desired light profile designed to control certain opsins 20.

As shown in FIG. 7d, a light source 26 is transmitting a first optical signal 81 which is received by both a first opsin site 83 and an optical conductor 82. When received by the first opsin site 83, the first optical signal 81 may trigger a physiological response, such as the generation of an action potential down a nerve cell. When received by the optical conductor 82, the first optical signal 81 may be received by the light modifying device 86 which will then modify the first optical signal 81 and/or generate a new signal to output a second optical signal 85 which is then received by a second opsin site 87. In this fashion, the first optical signal 81 may affect the second optical signal 85.

In some embodiments, the light modifying device 86 may contain a separate light source capable of supplementing the pre-optical-signal light 84 in the event the pre-optical-signal light 84 is insufficient to create the desired optical signal 24.

In some embodiments, the light modifying device 86 may contain an optical sensor and a light source and may be configured such that upon receiving a certain pre-optical-signal light 84, the light modifying device 86 may create an optical signal 24 solely from its own light source. In such embodiments, the light modifying device 86 does not actually modify the light passing through it, but effectively modifies the light since the pre-optical-signal light 84 that enters the light modifying device 86 is different from the optical signal 24 that exits it.

In some embodiments, the light modifying device 86 is a physical light barrier that can open and close (e.g., a douser or iris), or any other physical, chemical, or other device that is capable of altering the pre-optical-signal light 84 before it reaches the opsin site 80.

IV. Feedback

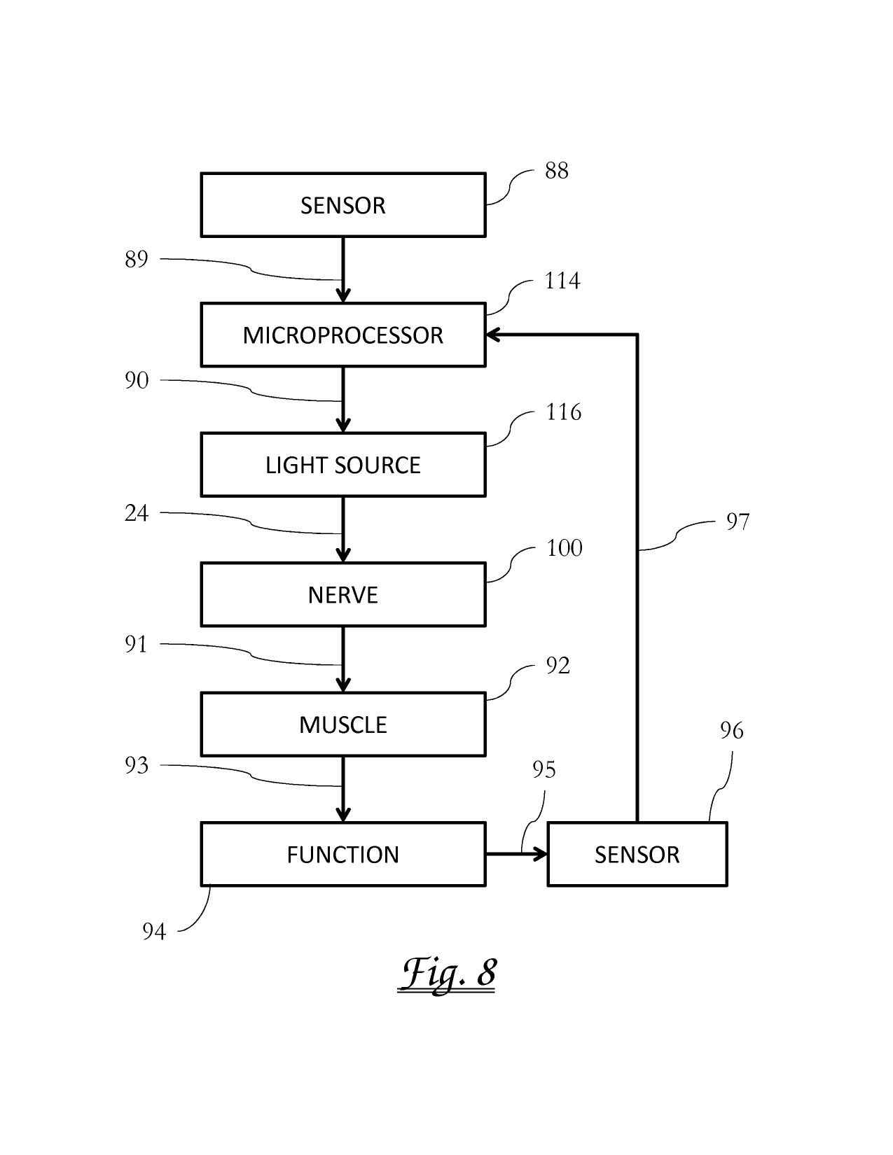

As shown in FIG. 8, some embodiments include a feedback system to provide better control over the optical signals necessary to control the targeted nerve or muscle in the desired fashion. In an embodiment, a sensor 88 may consist of an external switch, a wireless communication device, a physiological sensor, and/or another type of sensing device. The sensor 88 is capable of sending an initiation signal 89 to the microprocessor 114. In some embodiments, the sensor may be comprised of a handheld or wearable communications device upon which a user may initiate a command which is eventually received by the microprocessor 114 in the form of an initiation signal 89. If the initiation signal 89 calls for control of any opsin-affected nerves 100 or targeted muscles 92 to result in the performance of some function 94, then the microprocessor 114 will send an activation signal 90 to a light source 116 which will then cause an optical signal 24 to be applied to opsin-affected nerve 100. Upon application of the optical signal 24, the opsin-affected nerve 100 may become excited, causing an action potential to pass down the nerve. The presence and frequency of action potentials in the opsin-affected nerve 100 create a muscle activation signal 91 which is passed to the targeted muscle 92. If the muscle activation signal 91 is composed of a certain frequency of action potentials, the targeted muscle 92 will contract 93, resulting in performance or part-performance of a certain function 94. A sensor 96 then senses 95 the function 94 and transmits a feedback signal 97 to the microprocessor 114. The sensor 96 is selected to sense the performance of function 94. The microprocessor 114 contains programming capable of determining the desired activation signal 90 to send in response to the feedback signal 97 and/or the initiation signal 89. In some instances the desired activation signal 90 is a null signal, or one which results in no optical signal 24 being applied to the opsin-affected nerve 100. In other instances, the desired activation signal 90 sent may excite or inhibit the opsin-affected nerve 100. It will be understood that the embodiment described above and shown in FIG. 8 is simplified and that other embodiments may include multiple light sources 116, opsin-affected nerves, 100, targeted muscles 92, and desired functions 94. In some embodiments, a single microprocessor 114 may contain programming capable of sending distinct activation signals to distinct light sources upon reception of the initiation signal 89 and/or the feedback signal 97. In some embodiments, the microprocessor 114 may be capable of storing and/or transmitting data collected from sensor 88 and/or sensor 96.

It will be understood that instead of using a light source 116, as shown in FIG. 8, a light-modifying device may be used instead. Additionally, it will be understood that the opsin-affected nerve 100 as shown in FIG. 8 may be bypassed entirely if the targeted muscle 92 is affected by opsins. In such a case, the optical signal 24 may be applied directly to the targeted muscle 92. It will be understood that one or more nerve cells may be used to conduct the action potential to a neuromuscular junction. While the above description of the embodiment shown in FIG. 8 describes how the optical signal 24 results in excitation of the opsin-affected nerve 100, it will be understood that a certain activation signal 90 may be sent such that the resulting optical signal 24 further results in inhibition of the opsin-affected nerve 100. It will also be understood that sensor 96 may directly sense the contraction 93 of target muscle 92; the electrical and/or chemical environment in, on, or surrounding the target muscle 92; the electrical and/or chemical environment in, on, or surrounding the opsin-affected nerve 100; and/or any other variable that can be somehow correlated to the performance of the desired function 94.

Sensor 96 may be a single sensor or multiple sensors, of any type capable of providing the desired feedback. In an embodiment, the sensor 96 may be a force transducer capable of detecting contraction and/or movement of a muscle. Such a force transducer may be implanted in, on, or near a muscle or may be external to a body. Such a force transducer may be artificially created or may be natural (e.g., muscle spindles). In such an embodiment, the desired function to be controlled may be linked to the amount of contraction in a certain muscle. In this embodiment, one or more force transducers capable of detecting contraction and/or movement of one or more targeted muscles may provide one or more feedback signals to the microprocessor which then allow the microprocessor to adjust the one or more optical signals being transmitted to the one or more opsin-affected nerves to either decrease or increase the amount of contraction and/or movement of the one or more targeted muscles until desired levels of contraction and/or movement are reached.

In another embodiment, the sensor 96 may be comprised of one or more electrical sensors capable of detecting electrical activity of a nerve and/or muscle. In such an embodiment, the sensor 96 would provide one or more feedback signals to the microprocessor which then allow the microprocessor to adjust the one or more optical signals being transmitted to the one or more opsin sites to either decrease or increase the amount of electrical activity in the one or more opsin-affected nerves and/or targeted muscles until desired levels of electrical activity are reached.

Other embodiments may use other sensors, including but not limited to other electrical sensors, optical sensors, chemical sensors, and mechanical sensors. Other embodiments may use these other sensors and other feedback systems which are capable of providing a feedback signal 97 to the microprocessor 114 such that the microprocessor 114 can control the optical signal 24 applied to the opsin-affected nerve 100 such that the desired function 94 is controlled in the desired fashion.

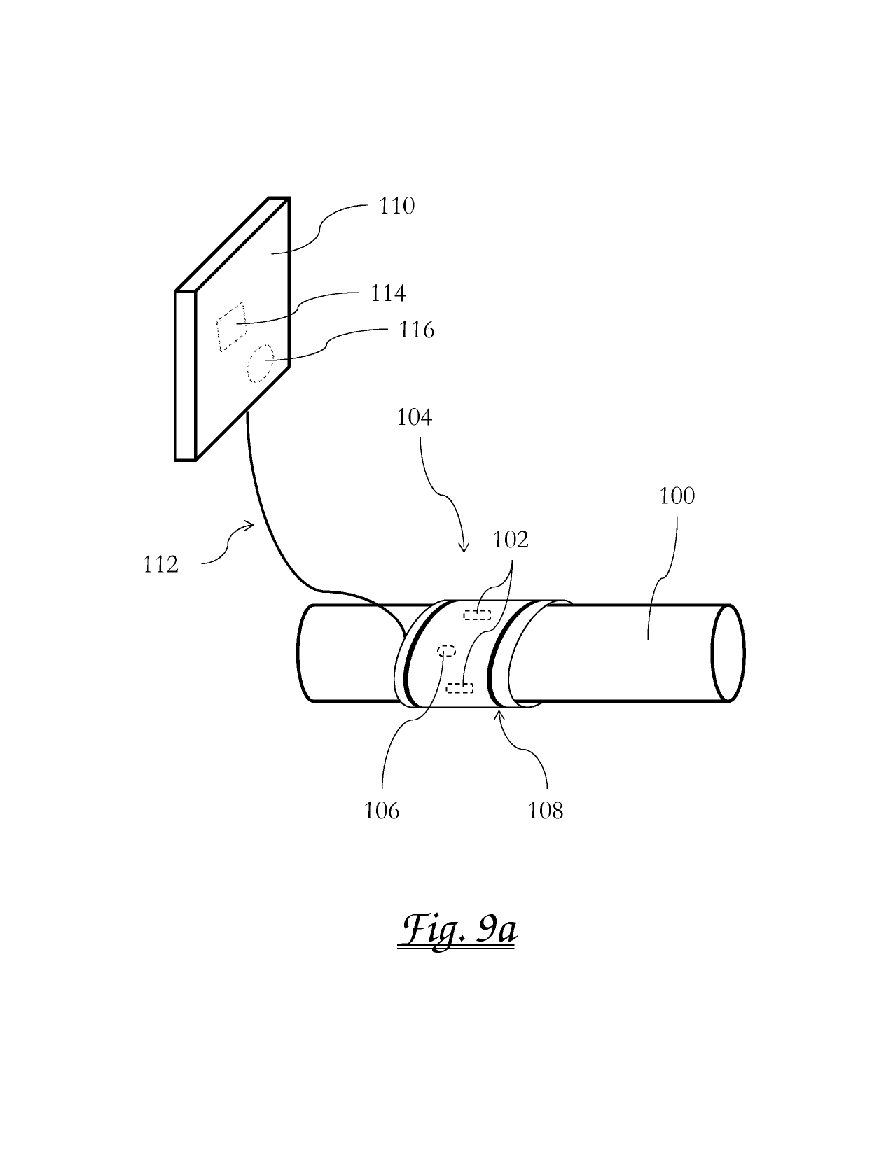

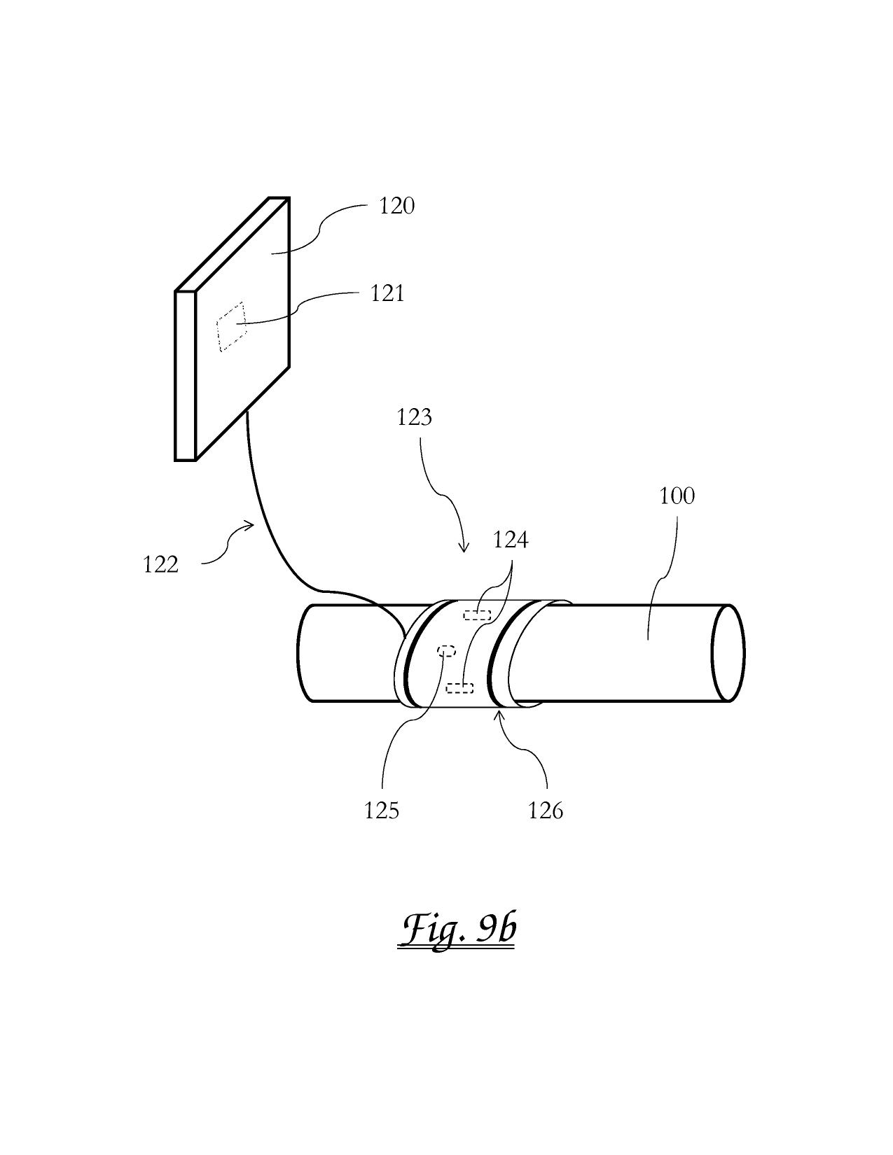

Another embodiment of the system and method for controlling neural and muscular function is shown in FIG. 9a. An implantable control device 110 containing a power source, a microprocessor 114, and a light source 116 is connected to a nerve cuff 104 by a cable 112. The cable 112 may contain multiple optical conductors and multiple electrical conductors, although any combination of optical conductors and electrical conductors may be used. The nerve cuff 104 is supported around a nerve bundle 100. The nerve cuff 104 may contain multiple contact electrodes 102 and multiple ring return electrodes 108, although different combinations of contact electrodes and ring return electrodes may be used. The electrodes 102, 108 of the nerve cuff 104 are connected to the microprocessor 114 through the electrical conductors of cable 112. The nerve cuff 104 additionally contains at least one optical signal output site 106. Optical signals from the light source 116 pass through the optical conductors of cable 112 and out the optical signal output sites 106 located in the nerve cuff 104. While one optical output site 106 is shown in FIG. 9a, it will be understood that many optical output sites 106 may be located in, on, or near the nerve cuff 104. Additionally, the nerve cuff 104 may itself be made of an optically conductive material, allowing the entire nerve cuff 104 to operate as an optical output site 106.

In some embodiments, multiple cables 112 and multiple nerve cuffs 104 may be used and may be connected to a single implantable control device 110. In some embodiments, multiple nerve cuffs 104 may be initially implanted or installed even if only certain nerve cuffs which have been installed at optimal locations are eventually used.

In some embodiments, the nerve cuff 104 may contain no optical output sites 106 and/or the cable 112 may contain no optical conductors, rather the optical signal is transmitted directly from the light source 116 through any number of optical conductors not located within the cable 112 or nerve cuff 104. In such embodiments, the optical conductors may include fluid or tissue surrounding the opsin site.

In some embodiments, the light source 116 is not located within the implantable control device 110. In some embodiments, the light source 116 may be located in, on, or near the nerve cuff 104 itself. Referring to FIG. 9b, an embodiment is shown wherein a nerve cuff 123 is situated on an opsin-affected nerve 100. An implantable control device 120 is shown containing a microprocessor 121, but no light source. A cable 122 containing electrical conductors connects the microprocessor 121 with the nerve cuff 123. The nerve cuff 123 contains contact electrodes 124, ring electrodes 126, and a light source 125. The light source 125 is positioned in, on, or near the nerve cuff 123 such that when it illuminates, the optical signal it creates is received at the opsin site of the opsin-affected nerve 100. Illumination of the light source 125 to produce the desired optical signals is achieved by the microprocessor 121 sending activation signals to the light source 125 along cable 112.

In some embodiments, the implantable control devices 110, 120 are capable of wirelessly communicating to and/or from an external control device, such as a physician's computer, for programming the implantable control devices 110, 120, for reporting data stored in or accessible to the implantable control devices 110, 120, and/or for other uses requiring transmission of data to and/or from the implantable control devices 110, 120.

In some embodiments, instead of using implantable control devices 110, 120, the transmission and/or generation of optic signals is controlled by control circuitry external to the body and communicating into and out of the body through conductors or wirelessly.

V. Exemplary Treatments

Treatment of Paralysis

Optogenetic intervention can be used to produce contraction in paralyzed muscles. Particularly, light application of specific and controlled intensities and durations to opsin sites located in or before a nerve cell attached to a paralyzed muscle can cause contraction of the paralyzed muscle. Careful control of light application to these opsin sites can restore useful function to people with paralysis of limbs and organs. Some of the best candidates for optogenetic treatment of paralysis include muscles which have become paralyzed as a result of central nervous system injury and disease, such as brain injury, spinal cord injury, stroke, or multiple sclerosis. Muscles paralyzed from other injuries or diseases or muscles which have been paralyzed since formation may be controlled by optogenetic treatment as disclosed above.

Treatment of Muscle Spasticity

Optogenetic intervention can be used to reduce the number or strength of contractions in muscles. Optogenetic intervention can therefore reduce muscle spasms or muscular spasticity. Particularly, careful application of light to certain opsin sites can control muscle spasticity with more specificity and with a more controlled duration than that which can be achieved through pharmaceutical or electrical interventions. Optogenetic treatment of muscle spasticity can improve function in people with stroke, brain injury, spinal cord injury, multiple sclerosis, cerebral palsy, or other conditions such as an overactive bladder.

Treatment of Pain

Optogenetic intervention can be used to control pain in living bodies. Careful application of light to certain opsin sites can be used to reduce the generation or conduction of electrical activity in nerves and nerve circuits involved in the perception of pain. Such optogenetic intervention can potentially relieve pain with more specificity and fewer side effects than existing medical and surgical treatments.

Control of Secretion

Optogenetic intervention can be used to control internal or external secretions in a living body. Careful application of light to certain opsin sites can reduce or increase secretions, whether internal (e.g. hormones, transmitters, releasing factors, trophins, cytokines, growth factors, etc) or external (e.g. digestive enzymes). Such optogenetic intervention may control the cardiovascular, gastrointestinal, respiratory, endocrine and other systems in addition to the neuromuscular system. Control of secretions allows optogenetic intervention to control and affect a number of other systems in a living body.

Treatment of Bladder, Bowel and Sexual Dysfunction

Numerous embodiments of neural and muscular control are described below, all of which may include opsins which can be placed and activated analogously as described below. In the following cases, where necessary, virus vectors containing the genes for encoding the desired opsins can be inserted via needles passed into: the bladder wall through a cystoscope; the external urethral sphincter through a urethroscope or the skin of the perineum; the internal urethral sphincter through a urethroscope or the skin of the perineum; the bowel wall through a proctoscope, sigmoidoscope, or colonoscope; the external anal sphincter through a proctoscope or anoscope; the internal anal sphincter through a proctoscope or anoscope; the pelvic floor muscles and nerves through the skin; the sacral afferent nerves by injection under the skin near those nerves; the sacral afferent or efferent nerves via injection into sacral foramina around these nerves; pudendal motor and sensory nerves via injection into or near these nerves; pelvic parasympathetic nerves via injection into or near the pelvic plexus; or the pelvic sympathetic nerves via injection into or near the hypogastric plexus or sympathetic trunks.

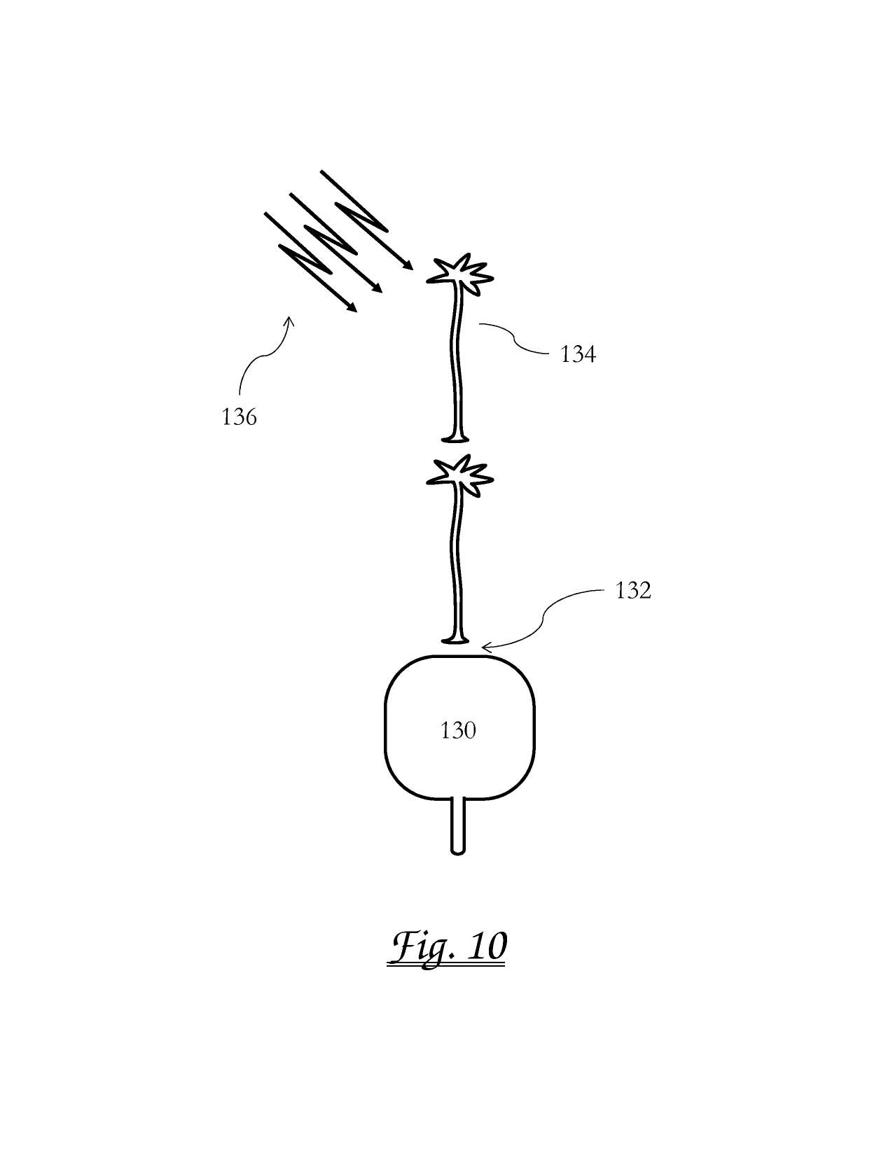

Continence of Urine: In an embodiment, the use of optogenetic intervention as described herein can reduce incontinence of urine. In one version of this embodiment, reduction in contraction of the bladder is achieved by using light to activate opsins which inhibit preganglionic or postganglionic parasympathetic efferent nerves. As shown in FIG. 10, viral vectors containing genes for inhibitory opsins are injected into the smooth muscle of the bladder wall 130 in some fashion, such as via cystoscopy. After allowing the vectors to transport the genes from the smooth muscle 130 across the neuromuscular junction 132 into preganglionic parasympathetic neurons 134, the genes will lead to production of opsins incorporated into the cellular membranes of the preganglionic parasympathetic neurons 134. Optical signals 136 can then be transmitted to the preganglionic parasympathetic neurons 134 as described above, including the use of light emitting devices (e.g., optrodes) or optical conductors (e.g. optical fibers, transparent tissues, translucent tissues, cerebrospinal fluid). In another version of this embodiment, reduction in contraction of the bladder can be achieved by using optical signals to activate opsins which stimulate sacral afferent nerves which thereby produce reflex inhibition of bladder contraction. In another version of this embodiment, contraction of the external urethral sphincter can be achieved by using optical signals to activate opsins which stimulate somatic efferent nerves which control the external urethral sphincter. In another embodiment, contraction of the internal urethral sphincter can be achieved by using optical signals to activate opsins which stimulate sympathetic efferent nerves which control the internal urethral sphincter.

Passing of Urine: In an embodiment, optogenetic intervention as described herein can improve the passing of urine. In one version of this embodiment, contraction of the bladder can be achieved by using optical signals to activate opsins which stimulate preganglionic parasympathetic efferent nerves which control contraction of the bladder. In another version of this embodiment, contraction of the bladder can be achieved by using optical signals to activate opsins which stimulate sacral afferent nerves which thereby produce reflex contraction of the bladder. In another version of this embodiment, reduced contraction of the external urethral sphincter can be achieved by using optical signals to activate opsins which inhibit somatic efferent nerves which control contraction of the external urethral sphincter. In another version of this embodiment, reduced contraction of the internal urethral sphincter can be achieved by using optical signals to activate inhibiting sympathetic efferent nerves which control the internal urethral sphincter.

Continence of Feces: In an embodiment, optogenetic intervention as described herein can reduce the incontinence of feces. In one version of this embodiment, a reduction in contraction of the bowel can be achieved by using optical signals to activate opsins which inhibit preganglionic parasympathetic efferent nerves which would otherwise trigger contraction of the bowel. In another version of this embodiment, a reduction in contraction of the bowel can be achieved by using optical signals to activate opsins which stimulate sacral afferent nerves which produce reflex inhibition of bowel contraction. In another version of this embodiment, contraction of the external anal sphincter can be achieved by using optical signals to activate opsins which stimulate somatic efferent nerves which produce contraction of the external anal sphincter. In another version of this embodiment, contraction of the internal anal sphincter can be achieved by using optical signals to activate opsins which stimulate sympathetic efferent nerves which produce contraction of the internal anal sphincter.

Passing of Feces: In an embodiment, optogenetic intervention as described herein can improve the passing of feces. In one version of this embodiment, contraction of the colon and rectum can be achieved by using optical signals to activate opsins which stimulate preganglionic parasympathetic efferent nerves which control contraction of the colon and rectum. In another version of this embodiment, contraction of the colon and rectum can be achieved by using optical signals to stimulate sacral afferent nerves which produce reflex contraction of the colon and rectum. In another version of this embodiment, reduced contraction of the external anal sphincter can be achieved by using optical signals to activate opsins which inhibit somatic efferent nerves which control the contraction of the external anal sphincter. In another version of this embodiment, reduced contraction of the internal anal sphincter can be achieved by using optical signals to activate opsins which inhibit sympathetic efferent nerves, thereby reducing contraction of the internal anal sphincter.

Reduction of Constipation: In an embodiment, optogenetic intervention as described herein can reduce constipation. In one version of this embodiment, increased transport of feces through the bowel can be achieved by using optical signals to activate opsins which stimulate preganglionic parasympathetic efferent nerves which control propulsive contractions or peristalsis of the colon. In another version of this embodiment, increased transport of feces through the bowel can be achieved by using optical signals to activate opsins which stimulate sacral afferent nerves which thereby produce reflex propulsive contractions or peristalsis of the colon.

Erection of the Penis: In an embodiment, optogenetic intervention as described herein can produce erection of the penis. In one version of this embodiment, increased blood flow to the corpora cavernosa of the penis can be achieved by using optical signals to activate opsins which stimulate preganglionic parasympathetic efferent nerves which control vasodilation of blood vessels to the corpora cavernosa. In another version of this embodiment, increased blood flow to the corpora cavernosa of the penis can be achieved by using optical signals to activate opsins which stimulate sacral afferent nerves which produce reflex vasodilation of blood vessels to the corpora cavernosa.

Emission of Semen: In an embodiment, optogenetic intervention as described herein can produce emission of semen. In one version of this embodiment, contraction of the prostate and seminal vesicles can be achieved by using optical signals to activate opsins which stimulate sympathetic efferent nerves which control contraction of the prostate and seminal vesicles. In another version of this embodiment, contraction of the prostate and seminal vesicles can be achieved by using optical signals to activate opsins which stimulate sacral afferent nerves which produce reflex contraction of the prostate and seminal vesicles.

Ejaculation of Semen: In an embodiment, optogenetic intervention as described herein can produce ejaculation of semen. In one version of this embodiment, contraction of the bulbospongiosus and ischiocavernosus muscles can be achieved by using optical signals to activate opsins which stimulate somatic efferent nerves which control contraction of the bulbospongiosus and ischiocavernosus muscles. In another version of this embodiment, contraction of the bulbospongiosus and ischiocavernosus muscles can be achieved by using optical signals to activate opsins which stimulate afferent nerves which produce reflex contraction of the bulbospongiosus and ischiocavernosus muscles.

Orgasm: In an embodiment, optogenetic intervention as described herein can produce orgasm. In one version of this embodiment, production of the sensation of orgasm and associated muscle contractions and secretions can be achieved by using optical signals to activate opsins which stimulate afferent nerves associated with the sensation and reflexes associated with orgasm.

It will be appreciated that many of the above embodiments can be combined to improve function and/or to produce multiple functions.

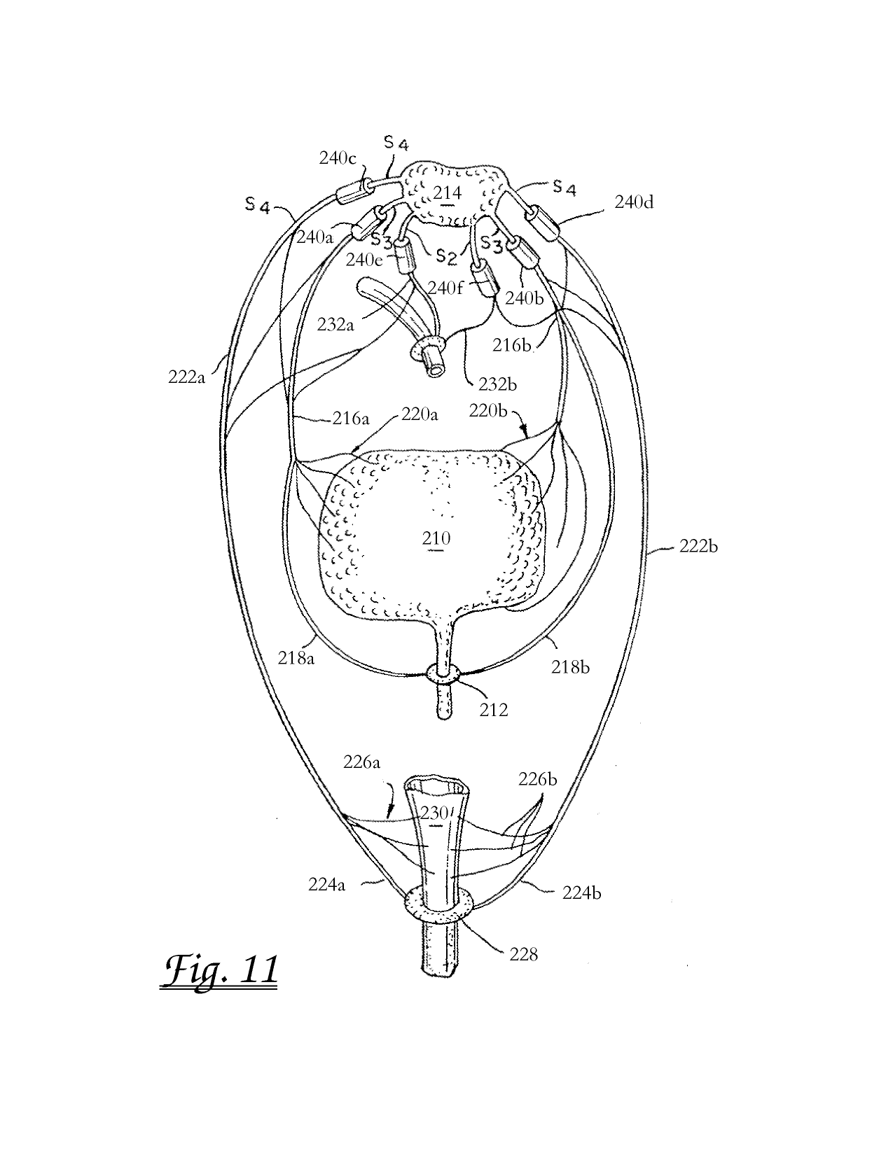

An embodiment of using optogenetic intervention to control multiple aspects of bladder, bowel, and sexual function is disclosed with reference to FIG. 11. Various lower abdominal and pelvic organs are controlled by action potentials that travel from the brain, through the spinal cord, and through sacral ventral roots to the end organs. More specifically, action potentials traveling along the S₂, S₃, and S₄ sacral ventral roots control continence of urine, bladder evacuation, continence of stool, bowel evacuation, constipation, penile erection, emission and ejaculation of semen, vaginal secretions, and orgasm.

Looking first to bladder evacuation, the bladder 210 and urethral sphincters 212 are controlled by action potentials traveling from the spinal cord 214 primarily, but not limited to, on a left-right symmetric pair of S₃ sacral ventral roots.

The S₃ ventral roots include bundles 216a, 216b of nerve fibers including larger diameter fibers 218a, 218b and smaller diameter fibers 220a, 220b. The larger diameter fibers connect between the spinal cord 214 and the external urethral sphincter 212. Action potentials flowing along the larger diameter nerve fibers cause the urethral sphincter to contract, blocking the outlet from the bladder 210. When the bladder is to be emptied, the flow of action potentials through the larger diameter nerve fibers is stopped allowing the sphincter to relax.

The smaller nerve fibers 220a, 220b connect between the spinal cord and the bladder, particularly the detrusor muscle which causes the bladder to contract. In a healthy person, the smaller diameter fibers usually carry no action potentials until the person desires to evacuate the bladder. To evacuate the bladder, action potentials are sent along the smaller diameter nerve fibers 220a, 220b concurrently with the stopping of sending action potentials along the larger diameter nerve fibers 218a, 218b. This causes the urethral sphincter to relax and allow the bladder outlet to open concurrently with detrusor contracting to expel urine.

Analogously, the S₃ and S₄ sacral ventral roots and to a lesser extent the S₂ sacral ventral roots provide nerve fibers which define bundles 222a, 222b of large diameter nerve fibers 224a, 224b and smaller diameter nerve fibers 226a, 226b. The large diameter nerve fibers control the external anal sphincter muscle 228 and the small diameter fibers 226a, 226b control muscles which cause contraction around the rectal canal 230. Defecation is accomplished by concurrently terminating the supply of action potentials to the anal sphincter 228 allowing it to relax while smaller diameter nerve fibers 226a, 226b carry action potentials to the muscles which cause the rectal canal 230 to contract.

Analogously, bundles of nerve fibers 232a, 232b primarily from the S₂ ventral roots control penile erection.

Spinal cord injuries and many other medical conditions can cause a loss of control of these organs. Optogenetic intervention can help reinstitute this control by introducing one or more opsins and one or more light signals to either activate or inactivate one or more types of nerve in one or more locations. Multiple opsins and multiple light signals can be introduced and combined for improved and specific function and/or multiple functions.

In an embodiment, a nerve cuff 240a-f, is mounted surrounding each of the appropriate sacral ventral roots and opsins are introduced according to the methods described above. Every nerve cuff 240a-f is individually configured to transmit appropriate optical signals to opsin sites located near the nerve cuff 240a-f, such that action potentials are generated or blocked according to the combinations of opsins and light signals introduced.

For example, for passing urine, nerve cuffs 240a, 240b surrounding the S₃ roots can generate action potentials in smaller diameter nerve fibers which activate the detrusor while blocking the transmission of action potentials on large diameter nerve fibers 218a, 218b to allow the external urethral sphincter 212 to relax. For continence of urine, a different light signal applied to the same nerve cuffs can generate action potentials in nerve fibers to the sphincter, causing it to contract, and block action potentials in nerve fibers to the bladder, allowing it to relax.

Analogously, nerve cuffs 240c, 240d are implanted around the S₄ roots to control defecation and fecal continence and other functions. Analogously, nerve cuffs 240e, 240f are implanted around the S2 roots to control penile erection and other functions.

In some embodiments, one or more nerve cuffs 240a-f may be placed along any of the large or small diameter nerve fibers 216a-b, 218a-b, 220a-b, 222a-b, 224a-b, 226a-b, 232a to elicit better control of the desired functions.

Analogously, specific opsins may be introduced into other smaller or larger nerve fibers, nerves and neural circuits and specific optical signals can be applied to specific nerve cuffs to control other functions.

Nerve fibers in the dorsal sacral roots carry afferent (sensory) signals which may produce reflex activation or inactivation of muscles and organs involved in bladder, bowel and sexual function. In some embodiments, one or more nerve cuffs may be placed along any of the dorsal sacral roots to affect opsins causing activation or inactivation of (afferent) sensory nerves, thereby influencing reflexes which affect bladder, bowel or sexual function.

Nerve fibers in the sympathetic nerves and trunks and the hypogastric plexus carry signals which may produce activation or inactivation of muscles and organs involved in bladder, bowel and sexual function. Opsins introduced into sympathetic nerves can be activated by light to improve functions affected by the sympathetic system

In some embodiments, light may be introduced into the cerebrospinal fluid surrounding the brain, spinal cord, cauda equina and spinal nerve roots and conducted through this transparent fluid to affect any or all of the nerves within this fluid into which opsins have been introduced.

It will be appreciated that other embodiments can be used in which opsins and light are applied to other nerves in the central or peripheral nervous system, such as the pudendal nerve and its branches and tributaries.

The above disclosure uses the examples of nerve cells and muscles to demonstrate the utility of optogenetic intervention in an animal. It will be understood that the same principles and disclosures set forth above may apply to the control of electrical activity and chemical activity in other cells of an animal simply by placing opsins in those cells and applying optical signals to those cells.

The headings used in this description are inserted for readability purposes only and are not to be construed as limiting, in any way, the contents of this disclosure.

As used in the claims below, the term “communicatively connected” refers to any connection, be it electrical, optical, wireless, or other, that allows for the transmission of data or signals between two objects. It is contemplated that two devices may be communicatively connected in various fashions, including by means of an electrical conductor for transferring an electrical signal and by means of a wireless connection transferring data packets.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

1. A method for controlling bladder, bowel, or sexual dysfunction comprising:
preparing first and second sets of opsin sites by introducing first and second pluralities of opsins responsive to first and second light profiles, at first and second opsin sites located on first and second neurons, via at least one of either synaptic or retrograde transfer of viral vectors from first and second introduction targets, respectively, the introduction targets including one or more of a patient's bladder wall, external urethral sphincter, internal urethral sphincter, bowel wall, external anal sphincter, internal anal sphincter, sacral afferent nerves, sacral efferent nerves, pudendal motor nerves, pudendal sensory nerves, pelvic parasympathetic nerves, and pelvic sympathetic nerves;
optically connecting one or more light sources to the first and second sets of opsin sites, the one or more light sources producing one or more optical signals to the first and second sets of opsin sites;
communicatively connecting one or more microprocessors to the one or more light sources to control the one or more light sources to produce the one or more optical signals; and
communicatively connecting a sensor to the one or more microprocessors, for transmitting a signal to at least one of the one or more microprocessors to control the one or more light sources.

2. The method of claim 1 wherein the one or more light sources is a single light source optically connected to the first and second opsin sites and the one or more microprocessors is a single microprocessor communicatively connected to the single light source.

3. The method of claim 2 wherein the first light profile is different from the second light profile and wherein the single microprocessor is configured to control the single light source to switch between the first light profile and the second light profile.

4. A method comprising:
introducing a plurality of opsins, at one or more opsin sites, into one or more cellular membranes of one or more nerve cells;
placing a light source in a location optically connected to at least one of the one or more opsin sites;
communicatively connecting a microprocessor to the light source to control the light source to produce an optical signal to excite or inhibit the one or more nerve cells; and
communicatively connecting a sensor to the microprocessor to transmit a control signal to the microprocessor to control the light source to produce the optical signal.

5. The method of claim 4 used to control bladder function, wherein:
the act of introducing the plurality of opsins at the one or more opsin sites comprises:
preparing a first set of opsin sites by performing one or more of:
preparing an inhibitory bladder wall opsin site with a plurality of inhibitory bladder wall opsins, the bladder wall opsin site being located on preganglionic parasympathetic nerves of bladder wall muscles;
preparing a bladder continence opsin site with a plurality of excitatory bladder continence opsins, the bladder continence opsin site being located on sacral afferent neurons that control reflex inhibition of bladder contraction;
preparing an excitatory external urethral sphincter opsin site with a plurality of excitatory external urethral sphincter opsins, the excitatory external urethral sphincter opsin site being located on somatic efferent nerves that control the external urethral sphincter; and
preparing an excitatory internal urethral sphincter opsin site with a plurality of excitatory internal urethral sphincter opsins, the excitatory internal urethral sphincter opsin site being located on sympathetic efferent nerves that control the internal urethral sphincter; and
preparing a second set of opsin sites by performing one or more of:
preparing an excitatory bladder wall opsin site with a plurality of excitatory bladder wall opsins, the excitatory bladder wall opsin site being located on preganglionic parasympathetic neurons of the bladder wall muscles;
preparing a bladder contraction reflex opsin site with a plurality of excitatory bladder contraction reflex opsins, the bladder contraction reflex opsin site being located on sacral afferent neurons that produce reflex contraction of the bladder;
preparing an inhibitory external urethral sphincter opsin site with a plurality of inhibitory external urethral sphincter opsins, the inhibitory external urethral sphincter opsin site being located on somatic efferent nerves that control an external urethral sphincter; and
preparing an inhibitory internal urethral sphincter opsin site with a plurality of inhibitory internal urethral sphincter opsins, the inhibitory internal urethral sphincter opsin site being located on sympathetic efferent nerves that control the internal urethral sphincter;
wherein the microprocessor is configured to reduce the incontinence of urine by activating the first set of opsin sites, and the microprocessor is configured to encourage passing of urine by activating the second set of opsin sites.

6. The method of claim 4 used to control bowel function, wherein:
the act of introducing the plurality of opsins at the one or more opsin sites comprises:
preparing a first set of opsin sites by performing one or more of:
preparing an inhibitory bowel contraction opsin site with a plurality of inhibitory bowel contraction opsins, the inhibitory bowel contraction opsin site being located on preganglionic parasympathetic efferent nerves that trigger contraction of the bowel;
preparing a bowel continence opsin site with a plurality of excitatory bowel continence opsins, the bowel continence opsin site being located on sacral afferent neurons that control reflex inhibition of bowel contraction;
preparing an excitatory external anal sphincter opsin site with a plurality of excitatory external anal sphincter opsins, the excitatory external anal sphincter opsin site being located on somatic efferent nerves that control the external anal sphincter; and
preparing an excitatory internal anal sphincter opsin site with a plurality of excitatory internal anal sphincter opsins, the excitatory internal anal sphincter opsin site being located on sympathetic efferent nerves that control the internal anal sphincter; and
preparing a second set of opsin sites by performing one or more of:
preparing an excitatory bowel contraction opsin site with a plurality of excitatory bowel contraction opsins, the excitatory bowel contraction opsin site being located on preganglionic parasympathetic neurons that control contraction of the colon and rectum;
preparing a bowel contraction reflex opsin site with a plurality of excitatory bowel contraction reflex opsins, the bowel contraction reflex opsin site being located on sacral afferent neurons that produce reflex contraction of the colon and rectum;
preparing an inhibitory external anal sphincter opsin site with a plurality of inhibitory anal sphincter opsins, the inhibitory external anal sphincter opsin site being located on somatic efferent nerves that control an external anal sphincter; and
preparing an inhibitory internal anal sphincter opsin site with a plurality of inhibitory internal anal sphincter opsins, the inhibitory internal anal sphincter opsin site being located on the sympathetic efferent nerves that control the internal anal sphincter;
wherein the microprocessor is configured to reduce the incontinence of feces by activating the first set of opsin sites, and the microprocessor is configured to encourage passing of feces by activating the second set of opsin sites.

7. The method of claim 4 used to reduce constipation, wherein:
the act of introducing the plurality of opsins at one or more opsin sites comprises performing one or more of:
preparing a colon peristalsis opsin site with a plurality of excitatory colon peristalsis opsins, the colon peristalsis opsin site being located on preganglionic parasympathetic efferent nerves that control propulsive contractions or peristalsis of the colon; and
preparing a colon reflex opsin site with a plurality of excitatory colon reflex opsins, the colon reflex opsin site being located on sacral afferent neurons that control reflex propulsive contractions or peristalsis of the colon;
wherein the microprocessor is configured to reduce constipation by activating the one or more opsin sites.

8. The method of claim 4 used to control penile erection, wherein:
the act of introducing the plurality of opsins at one or more opsin sites comprises performing one or more of:
preparing a vasodilation opsin site with a plurality of excitatory vasodilation opsins, the vasodilation opsin site being located on preganglionic parasympathetic efferent nerves which control vasodilation of blood vessels to the corpora cavernosa; and
preparing a vasodilation reflex opsin site with a plurality of excitatory vasodilation reflex opsins, the vasodilation reflex opsin site being located on sacral afferent neurons which produce reflex vasodilation of blood vessels to the corpora cavernosa;
wherein the microprocessor is configured to increase blood flow to the corpora cavernosa by activating the one or more opsin sites.

9. The method of claim 4 used to control semen emission and ejaculation and orgasm, wherein:
the act of introducing the plurality of opsins at the one or more opsin sites comprises:
preparing a first set of opsin sites by performing one or more of:
preparing an semen emission opsin site with a plurality of excitatory semen emission opsins, the semen emission opsin site being located on sympathetic efferent nerves that control contraction of the prostate and seminal vesicles; and
preparing a semen emission reflex opsin site with a plurality of semen emission reflex opsins, the semen emission reflex opsin site being located on sacral afferent neurons that produce reflex contraction of the prostate and seminal vesicles;
preparing a second set of opsin sites by performing one or more of:
preparing a semen ejaculation opsin site with a plurality of excitatory semen ejaculation opsins, the semen ejaculation opsin site being located on somatic efferent nerves that control contraction of the bulbospongiosus and ischiocavernosus muscles; and
preparing a semen ejaculation reflex opsin site with a plurality of excitatory semen ejaculation reflex opsins, the semen ejaculation reflex opsin site being located on afferent nerves that produce reflex contraction of the bulbospongiosus and ischiocavernosus muscles; and
preparing a third set of opsin sites be performing one or more of:
preparing an orgasm opsin site with a plurality of excitatory orgasm opsins, the orgasm opsin site being located on afferent nerves associated with the sensation and reflexes associated with orgasm; and
wherein the microprocessor is configured to produce emission of semen by activating the first set of opsin sites, the microprocessor is configured to produce ejaculation of semen by activating the second set of opsin sites, and the microprocessor is configured to produce orgasm by activating the third set of opsin sites.

10. A method comprising:
preparing an opsin site located on a nerve by introducing a plurality of opsins via at least one of either synaptic or retrograde transfer of viral vectors from an introduction target, the introduction target including one of a patient's bladder wall, external urethral sphincter, internal urethral sphincter, bowel wall, external anal sphincter, internal anal sphincter, sacral afferent nerves, sacral efferent nerves, pudendal motor nerves, pudendal sensory nerves, pelvic parasympathetic nerves, and pelvic sympathetic nerves;
optically connecting a light source to the opsin site, the light source producing an optical signal to the opsin site;
communicatively connecting a microprocessor to the light source to control the light source to produce the optical signal.

11. The method of claim 10 used to effect bladder function, wherein:
the act of preparing the opsin site comprises performing one of:
preparing an inhibitory bladder wall opsin site with a plurality of inhibitory bladder wall opsins, the bladder wall opsin site being located on preganglionic parasympathetic nerves of bladder wall muscles;
preparing a bladder continence opsin site with a plurality of excitatory bladder continence opsins, the bladder continence opsin site being located on sacral afferent neurons that control reflex inhibition of bladder contraction;
preparing an excitatory external urethral sphincter opsin site with a plurality of excitatory external urethral sphincter opsins, the excitatory external urethral sphincter opsin site being located on somatic efferent nerves that control the external urethral sphincter;
preparing an excitatory internal urethral sphincter opsin site with a plurality of excitatory internal urethral sphincter opsins, the excitatory internal urethral sphincter opsin site being located on sympathetic efferent nerves that control an internal urethral sphincter;
preparing an excitatory bladder wall opsin site with a plurality of excitatory bladder wall opsins, the excitatory bladder wall opsin site being located on preganglionic parasympathetic neurons of the bladder wall muscles;
preparing a bladder contraction reflex opsin site with a plurality of excitatory bladder contraction reflex opsins, the bladder contraction reflex opsin site being located on sacral afferent neurons that produce reflex contraction of the bladder;
preparing an inhibitory external urethral sphincter opsin site with a plurality of inhibitory external urethral sphincter opsins, the inhibitory external urethral sphincter opsin site being located on somatic efferent nerves that control an external urethral sphincter; and
preparing an inhibitory internal urethral sphincter opsin site with a plurality of inhibitory internal urethral sphincter opsins, the inhibitory internal urethral sphincter opsin site being located on sympathetic efferent nerves that control an internal urethral sphincter;
wherein the microprocessor is configured to effect the incontinence of urine by activating the opsin site.

12. The method of claim 10 used to effect bowel function, wherein:
the act of preparing the opsin site comprises performing one of:
preparing an inhibitory bowel contraction opsin site with a plurality of inhibitory bowel contraction opsins, the inhibitory bowel contraction opsin site being located on preganglionic parasympathetic efferent nerves that trigger contraction of the bowel;
preparing a bowel continence opsin site with a plurality of excitatory bowel continence opsins, the bowel continence opsin site being located on sacral afferent neurons that control reflex inhibition of bowel contraction;
preparing an excitatory external anal sphincter opsin site with a plurality of excitatory external anal sphincter opsins, the excitatory external anal sphincter opsin site being located on somatic efferent nerves that control the external anal sphincter;
preparing an excitatory internal anal sphincter opsin site with a plurality of excitatory internal anal sphincter opsins, the excitatory internal anal sphincter opsin site being located on sympathetic efferent nerves that control an internal anal sphincter;
preparing an excitatory bowel contraction opsin site with a plurality of excitatory bowel contraction opsins, the excitatory bowel contraction opsin site being located on preganglionic parasympathetic neurons that control contraction of the colon and rectum;
preparing a bowel contraction reflex opsin site with a plurality of excitatory bowel contraction reflex opsins, the bowel contraction reflex opsin site being located on sacral afferent neurons that produce reflex contraction of the colon and rectum;
preparing an inhibitory external anal sphincter opsin site with a plurality of inhibitory anal sphincter opsins, the inhibitory external anal sphincter opsin site being located on somatic efferent nerves that control an external anal sphincter; and
preparing an inhibitory internal anal sphincter opsin site with a plurality of inhibitory internal anal sphincter opsins, the inhibitory internal anal sphincter opsin site being located on the sympathetic efferent nerves that control an internal anal sphincter;
wherein the microprocessor is configured to effect the incontinence of feces by activating the opsin site.

13. The method of claim 10 used to reduce constipation, wherein:
the act of preparing the opsin site comprises performing one of:
preparing a colon peristalsis opsin site with a plurality of excitatory colon peristalsis opsins, the colon peristalsis opsin site being located on preganglionic parasympathetic efferent nerves that control propulsive contractions or peristalsis of the colon; and
preparing a colon reflex opsin site with a plurality of excitatory colon reflex opsins, the colon reflex opsin site being located on sacral afferent neurons that control reflex propulsive contractions or peristalsis of the colon;
wherein the microprocessor is configured to reduce constipation by activating the opsin site.

14. The method of claim 10 used to control penile erection, wherein:
the act of preparing the opsin site comprises performing one of:
preparing a vasodilation opsin site with a plurality of excitatory vasodilation opsins, the vasodilation opsin site being located on preganglionic parasympathetic efferent nerves which control vasodilation of blood vessels to the corpora cavernosa; and
preparing a vasodilation reflex opsin site with a plurality of excitatory vasodilation reflex opsins, the vasodilation reflex opsin site being located on sacral afferent neurons which produce reflex vasodilation of blood vessels to the corpora cavernosa;
wherein the microprocessor is configured to increase blood flow to the corpora cavernosa by activating the opsin site.

15. The method of claim 10 used to control semen emission, wherein:
the act of preparing the opsin site comprises performing one of:
preparing an semen emission opsin site with a plurality of excitatory semen emission opsins, the semen emission opsin site being located on sympathetic efferent nerves that control contraction of the prostate and seminal vesicles; and
preparing a semen emission reflex opsin site with a plurality of semen emission reflex opsins, the semen emission reflex opsin site being located on sacral afferent neurons that produce reflex contraction of the prostate and seminal vesicles; and
wherein the microprocessor is configured to effect the emission of semen by activating the opsin site.

16. The method of claim 10 used to control semen ejaculation, wherein:
the act of preparing the opsin site comprises performing one of:
preparing a semen ejaculation opsin site with a plurality of excitatory semen ejaculation opsins, the semen ejaculation opsin site being located on somatic efferent nerves that control contraction of the bulbospongiosus and ischiocavernosus muscles;
preparing a semen ejaculation reflex opsin site with a plurality of excitatory semen ejaculation reflex opsins, the semen ejaculation reflex opsin site being located on afferent nerves that produce reflex contraction of the bulbospongiosus and ischiocavernosus muscles; and
wherein the microprocessor is configured to effect the ejaculation of semen by activating the opsin site.

17. The method of claim 10 used to control semen emission and ejaculation and orgasm, wherein:
the act of preparing the opsin site comprises preparing an orgasm opsin site with a plurality of excitatory orgasm opsins, the orgasm opsin site being located on afferent nerves associated with the sensation and reflexes associated with orgasm; and
wherein the microprocessor is configured to effect the production of orgasm by activating the opsin site.
